(19) United States
(12) Patent Application Publication
(10) Pub. No.: US 2021/0277481 A1
(43) Pub. Date: Sep. 9, 2021

(54) CIRCULATING TUMOR AND TUMOR STEM CELL DETECTION USING GENOMIC SPECIFIC PROBES

(71) Applicant: Board of Regents, The University of Texas System, Austin, TX (US)

(72) Inventor: Ruth KATZ, Houston, TX (US)

(73) Assignee: Board of Regents, The University of Texas System, Austin, TX (US)

(21) Appl. No.: 17/061,412

(22) Filed: Oct. 1, 2020

Related U.S. Application Data
(63) Continuation of application No. 15/534,599, filed on Jun. 9, 2017, now abandoned.
(60) Provisional application No. 62/090,167, filed on Dec. 10, 2014.

(51) Int. Cl. C12Q 1/6886; G01N 33/574

(52) U.S. Cl. CPC C12Q 1/6886; G01N 33/57488; C12Q 2600/156; C12Q 2600/112; G01N 2800/52; G01N 2800/56; C12Q 2600/118

(57) ABSTRACT

The present disclosure comprises a method of detecting circular tumor cells and methods of detecting, evaluating, or staging cancer in a patient, as well as a method of monitoring treatment of cancer in a patient using the claimed method. In other embodiments, the method provides for directed to a method of determining the level of circulating tumor cells (CTCs) in a sample having blood cells from a patient by contacting a sample having blood cells from a patient.

This application is a continuation of U.S. application Ser. No. 15/534,599, filed Jun. 9, 2017, which is a national phase application under 35 U.S.C. § 371 of International Application No. PCT/US2015/065057, filed Dec. 10, 2015, which claims benefit of priority to U.S. Provisional Application Ser. No. 62/090,167, filed Dec. 10, 2014, the entire contents of each of which are hereby incorporated by reference.

### BACKGROUND

### 1. Field

The present disclosure relates to the fields of oncology, genetics and molecular biology. More particularly, the disclosure relates to the use of probes for regions that are highly predictive of the development of neoplasia and progression of neoplastic events. Using this disclosure, subjects can be screened for, e.g., lung cancer using a minimal amount of blood (e.g., a finger prick).

### 2. Description of Related Art

In 2005, it is estimated that lung cancer accounted for 13% of new cancer cases and was the leading cause of cancer deaths in the United States. Unfortunately, the overall 5-year survival rate remains less than 15%, despite advances in treatment. Clearly, there is a need to develop novel strategies for treatment of lung cancer, and at the same time develop sensitive surrogate biomarkers that can serve to monitor early response to new therapies. The presence of circulating cancer cells (CTCs) or tumor stem cells that compose a small but vital part of the tumor subpopulation is presently considered to be the “holy grail” for detection and eradication for patient response and survival.

Cristofanilli et al. (2004), in a prospective study of patients with metastatic breast cancer, showed that patients whose CTCs were above 5 per 7.5 ml of blood at baseline were associated with both a significantly shorter progression-free survival and shorter overall survival. Pierga et al. similarly reported that the presence of cytokeratin positive CTCs in peripheral blood of patients with breast cancer corresponded with stage and prognosis (Pierga et al., 2004). Some investigators have looked at the genomic signatures in the metastasizing cells compared to the primary tumors and have found a gene expression signature in the primary tumor that predicts for metastasis and poor clinical outcome (Gangnus et al., 2004; Ramaswamy et al., 2003; Muller and Pantel, 2004). Others have used PCR to identify genes associated with CTCs in peripheral blood in non-small cell lung cancer (NSCLC) cases and have shown that poor therapeutic response was associated with detection of CTC after therapy (Sher et al., 2005).

A consensus is emerging that a crucial early event in carcinogenesis is the induction of the genomic instability phenotype, which enables an initiated cell to evolve into a cancer cell by achieving a greater proliferative capacity (Fenech et al., 2002). It is well known that cancer results from an accumulation of multiple genetic changes that can be mediated through chromosomal changes and therefore has the potential to be cytogenetically detectable (Solomon et al., 1991). It has been hypothesized that the level of genetic damage in peripheral blood lymphocytes reflects amount of damage in the precursor cells that lead to the carcinogenic process in target tissues (Hagmar et al., 1998). Evidence that cytogenetic biomarkers are positively correlated with cancer risk has been strongly validated in recent results from both cohort and nested case-control studies showing that chromosome aberrations as a marker of cancer risk (Liou et al., 1999; Bonassi et al., 2000; Bonassi et a., 2004; Smerhovsky et al., 2001; Tucker and Preston, 1996) reflecting both the genotoxic effects of carcinogens and individual cancer susceptibility commonly used methods for measuring DNA damage because it is relatively easier to score micronuclei (MN) than chromosome aberrations (Fenech et al., 2002). MN originates from chromosome fragments or whole chromosomes that fail to engage with the mitotic spindle and therefore lag behind when the cell divides. Compared to other cytogenetic assays, quantification of MN confer several advantages, including speed and ease of analysis, no requirement for metaphase cells and reliable identification of cells that have completed only one nuclear division, which prevents confounding effects caused by differences in cell division kinetics because expression of MN, NPBs or NBUDs is dependent on completion of nuclear division (Fenech, 2000). Because cells are blocked in the binucleated stage, it is also possible to measure nucleoplasmic bridges (NPBs) originating from asymmetrical chromosome rearrangements and/or telomere end fusions (Umegaki et al., 2000; Stewenius et al., 2005). NPBs occur when the centromeres of dicentric chromosomes or chromatids are pulled to the opposite poles of the cell at anaphase. In the CBMN assay, binucleated cells with NPBs are easily observed because cytokinesis is inhibited, preventing breakage of the anaphase bridges from which NPBs are derived, and thus the nuclear membrane forms around the NPB. Both MN and NPBs occur in cells exposed to DNA-breaking agents (Stewenius et al., 2005; Fenech and Crott, 2002) In addition to MN and NPBs, the CBMN assay allows for the detection of nuclear buds (NBUDs), which represent a mechanism by which cells remove amplified DNA and are therefore considered a marker of possible gene amplification (reviewed by Fenech (2002). The CBMN test is slowly replacing the analysis of chromosome aberrations in lymphocytes because MN, NPBs and NBUDs are easy to recognize and score and the results can be obtained in a shorter time (Fenech, 2002).

Factors predicting clinical outcome in lung cancer patients include extent of disease or tumor burden. Circulating tumor cells (CTCs) may be a measure of tumor burden, and may also be a method to more accurately stage patients. Previously CTCs were isolated from whole blood based on assays employing magnetic beads coated with anti-cytokeratin antibodies (positive selection) or depletion of CD45 lymphoid cells with an antibody to keratin (EPICAM) for epithelial cells or depletion of CD45 cells. The OncoQuick system involves gradient separated cells and immunohistochemistry followed by image analysis.

Other methods for obtaining CTCs include dielectrophoresis (DEP) array methods include where an enriched sample of cells containing fluorescence-labelled CTCs is injected into a cartridge through an array of electrodes, which generates a dielectric field. This traps each cell into a DEP virtual cage, whereafter cells of interest, based on their fluorescent pattern, can be selected and dispatched for further investigation. However, in order to discover which cells are of interest that may be CTCs, the operator has to have upfront knowledge of the immunophenotyping of the CTCs.

Previous methods to detect CTCs also include PCR-assays. However these cannot quantify number of tumor cells or look at morphology. Based on a previous methodology which was antigen-dependent and required that the CTCs express epCAM, an epithelial antigen expressed by only few CTCs in the blood stream, it was found that yields of circulating cancer cells have been low to absent, especially in lung cancer, and triple-negative breast cancer. Thus, there is a need to develop more sensitive and antigen agnostic methods for detecting CTCs and determining the level of CTCs in samples. One of the most promising ways to do this is to rely on a test that takes into account chromosomal abnormalities that are never found in normal peripheral blood mononuclear cells (PBMNCs) that define an aneuploid cell and that can be quantitated by inter-phase fluorescence-in-situ hybridization or FISH.

### SUMMARY

Provided is a method of detecting circulating tumor cells (CTCs) in a sample containing blood cells comprising (a) selecting CTCs from a sample containing blood cells by assessing nuclear area and/or circularity; (b) hybridizing the selected cells with labeled nucleic acid probes for 3p22.1, 10q22.3, chromosome 10 centromeric (cep10) and chromosome 3 centromeric (cep3); (c) evaluating the signal pattern for the selected cells by detecting fluorescence in situ hybridization from cells; and (d) detecting CTCs based on pattern of hybridization to all four labeled nucleic acid probes to said selected cells. The method may further comprise obtaining said sample. Step (d) may comprise assessing all abnormalities or gains only. The method may comprise abnormalities in two or more of the four probe set. The method may also employ detection CTC by detection of of a surfactant protein, such as SP-A, SP-B and/or SP-C, for example using immunohistochemistry.

Typically, the nuclei are stained in order to permit assessment/sorting, such as with DAPI (4′,6-diamidino-2-phenylindole). In certain embodiments, the nuclei will be obtained from cells and sorted on their own. Cells may be lysed using standard cells lysis protocols. A color or monochromatic CCD cameras normally images and classifies all nucleated cells presented on the cytopreparation. The number of cells classified is preset by the operator however usually several thousand cells are scanned for:
1) nuclear area in pixels, based on the DAPI stain, expressed as arbitrary units, thus, if 5000 it means that the cell area is 5000 pixels;
2) nuclear diameter; and
3) circularity factor (CFs), calculated by modifying the elongation (proportion between the height and the width of the cell) where a perfect circle will have the value of 1 (lymphocytes have CFs close to 1, abnormal cells have much CFs>>1, due to their nuclear perimeter irregularity).

The nuclear area for the abnormal (malignant CTCs) cells is based on the number of pixels occupied by the nucleus (as defined by FISH polysomy >2) as measured on the DAPI stain (a nuclear stain) and was expressed in arbitrary units. In embodiments where absolute numbers of CTCs are diagnostic, a finding of 4 or more CTCs will indicate that the patient has cancer.

The method may further comprise, prior to step (b), filtering said blood sample, such as by use of a vacuum apparatus and a membrane perforated with 7.5 μm pores, and further, the blood sample is a gradient separated sample of peripheral blood mononuclear cells. The blood sample may be a buffy coat layer separated from the blood by a Ficoll-Hypaque gradient, such as one that is further purified by CD3 and/or CD45 bead-based purification to remove white blood cells. Selecting CTCs may be achieved by assessing nuclear area comprises determining pixel size for each CTC and applying a pre-determined threshold for exclusion, by determining nuclear diameter, or by DAPI concentration and its standard deviation.

The patient may be known or suspected to have cancer, such as a form of cancer that gives rise to blood borne metastases, including but not limited to cancer of lung, head & neck, breast, colon, prostate, pancreas, esophagus, kidney, gastro-intestinal tumors, urigenital tumors, kidney, melanomas, endocrine tumors (thyroid including papillary thyroid cancer, adrenal gland cortex or medulla) or sarcomas. The staining may further comprise contacting the sample with a labeled CD45 antibody, a labeled SNAIL1 antibody, and/or a labeled anti-GLUT1 antibody, such as where the label is a fluorescent label or a chromagen label. Detecting of the signal may comprise using an automated fluorescence scanner.

The method may further comprise using and detecting one or more additional probes in steps (b)-(d), such as a UroVysion DNA probe set, LaVysion DNA probe set, a centromeric 7/7p12 Epidermal Growth Factor (EGFR) probe, cep7/7p22.1, cep17, and 9p21.3 probes, EGFR/cep and 10/cep10q probes, pTEN, cep10 and cep10q probes, and/or an EML4-ALK probe set.

In another embodiment, there is provided a method of determining the level of circulating tumor cells (CTCs) in a sample containing blood cells from a patient by (a) selecting CTCs from a blood sample by assessing nuclear size and/or circularity; (b) contacting the selected cells with labeled nucleic acid probes for 3p22.1, 10q22.3, chromosome 10 centromeric (cep10) and chromosome 3 centromeric (cep3); (c) detecting fluorescence in situ hybridization from cells; and (d) quantifying CTCs based on hybridization to all four labeled nucleic acid probes. Step (d) may comprise assessing all abnormalities or gains only. The method may comprise abnormalities in two or more of the four probe set. The method may also employ detection CTC by detection of of a surfactant protein, such as SP-A, SP-B and/or SP-C, for example using immunohistochemistry.

In yet another embodiment, there is provided a method of detecting cancer in a patient comprising determining the level of circulating tumor cells (CTCs) in a sample containing blood cells from the patient by any of the methods set out above, wherein the presence of CTCs equaling 4 or more in the sample is indicative of cancer, such as wherein the sample is a 5 ml sample of a separated buffy coat layer.

In still yet another embodiment, there is provided a method of detecting cancer in a patient comprising determining the level of CTCs in a biological sample containing blood cells from the patient by any of the methods set out above, wherein the presence of CTCs in the blood sample, in the presence of an indeterminate nodule of greater than 3 mm in the lung, is indicative of cancer.

In still a further embodiment, there is provided a method of screening for lung cancer in a patient at high risk for lung cancer, comprising determining the level of circulating tumor cells (CTCs) in a sample containing blood cells from the patient by any of the methods set out above, wherein the presence of CTCs in the blood sample is indicative of lung cancer. The high risk may be based on age >55 years, history of being a current or former smoker, exposure to second hand cigarette smoke, or having a family history of cancer. The method may further comprise performing a spiral CT scan when the presence of CTCs is observed. The method may further comprise repeating the methods at a second point in time to determine an increase in the level of CTCs.

In still yet another embodiment, there is provided a method of evaluating cancer in a patient comprising determining the level of circulating tumor cells (CTCs) in a sample containing blood cells from the patient by any of the methods set out above, wherein a higher level of CTCs in the sample, as compared to a control or predetermined number of CTCs from a non-aggressive form of cancer, is indicative of an aggressive form of cancer and/or a poor cancer prognosis.

The control may be a non-cancerous sample. The method may further comprise obtaining a patient sample, reporting the level of CTCs, and/or treating the cancer based on whether the level of CTCs is high, such as with chemotherapy, radiotherapy, surgery, gene therapy, immunotherapy, targeted therapy, or hormonal therapy.

In another embodiment, there is provided a method of monitoring treatment of cancer in a patient comprising (a) determining the level of CTCs in a first sample from the patient by any of the methods set out above; (b) determining the level of CTCs in a second sample from the patient after treatment is effected by any of the methods set out above; and (c) comparing the level of CTCs in the first sample with the level of CTCs in the second sample to assess a change, thereby monitoring treatment.

The method may further comprise continuing treatment if the the level of CTCs is reduced in step (b) as compared to step (a). The treatment may be chemotherapy, radiotherapy, surgery, gene therapy, immunotherapy, targeted therapy, or hormonal therapy. The method may further comprise discontinuing treatment if the the level of CTCs is not reduced in step (b) as compared to step (a). The method may further comprise obtaining said first and/or second patient samples.

Another embodiment comprises a method of staging cancer in a patient comprising determining circulating tumor cells (CTC) in a sample containing blood cells from the patient by any of the methods set out above, wherein a higher level of CTCs in the sample as compared to a predetermined control for a given stage is indicative of a more advanced stage of cancer, and a lower level of CTCs in the sample as compared to a control for a given stage is indicative of a less advanced stage of cancer.

The control may be a lung cancer stage 0 sample, a lung cancer stage I sample, a lung cancer stage 1A sample, a lung cancer stage IB sample, a lung cancer stage II sample, a lung cancer stage III sample, a lung cancer stage IV sample, and/or a lung non-cancerous sample. The method may further comprising obtaining a patient sample, reporting the level of CTCs, and/or treating the cancer if the level of CTCs is indicative of a more advanced stage of cancer. The treatment may be chemotherapy, radiotherapy, surgery, gene therapy, immunotherapy, targeted therapy, or hormonal therapy. The method may be used to refine the staging of cancer after treatment has started.

In particular embodiments, the level of CTCs is at least 50% more, compared to the level in a control sample. In other embodiments, the level of CTCs is at least about or at most about 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-fold or times, or any range derivable therein, greater than the level of a control sample. In particular embodiments, the level of CTCs is at least 2-fold greater than the level of a control sample.

For any of the preceding methods, one may also examine the level of expression of one or more cancer gene markers for alterations from the norm. These can further diagnosis, staging or prognosis of the methods.

It is contemplated that any embodiment discussed in this specification can be implemented with respect to any method or composition of the disclosure, and vice versa. Furthermore, compositions of the disclosure can be used to achieve methods of the disclosure.

The use of the word “a” or “an” in the claims and/or the specification may mean “one,” but it is also consistent with the meaning of “one or more,” “at least one,” and “one or more than one.”

The phrase “one or more” as found in the claims and/or the specification is defined as 1, 2, 3, 4, 5, 6, 7, 8, 9, 10, or more.

Throughout this application, the terms “about” and “approximately” indicate that a value includes the inherent variation of error for the device, the method being employed to determine the value, or the variation that exists among the study subjects. In one non-limiting embodiment the terms are defined to be within 10%, preferably within 5%, more preferably within 1%, and most preferably within 0.5%.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure may be better understood by reference to one or more of these drawings in combination with the detailed description of specific embodiments presented herein.

FIG. 1. Data flow.

FIGS. 2A-D. 3D scatter plot of 74 samples. FIG. 2A (top left), FIG. 2B (top right), FIG. 2C (bottom left) and FIG. 2D (bottom right)—3D scatter plot of 74 samples.

FIG. 3. Estimated number of clusters detected in data.

FIG. 4. Clustered samples using clustering method Average Linkage.

FIG. 5. EuclideanD (v-axis) vs Clusters (x-axis). The 2 diamonds (cases) are in the group of controls, and 3 larger circles in cluster 2 are the control samples. The arrow points to the two ambiguous samples with the same EuclideanD=859 (see Table 13).

FIG. 6. Genetic abnormalities in peripheral blood mononuclear cells evaluating gains and losses of chromosomes in all cells. Comparison between all genetic abnormalities, deletions and gains, between cases and controls in 500 peripheral blood cells, note that at a threshold of 25, we have 2 false positives but 100% sensitivity, if one combines with the other test for gains one gets almost completely perfect sensitivity and specificity. Y axis=abnormalities, x axis=number of subjects.

FIG. 7. Aneuploidy in circulating tumor cells defined as a cell with 2 or more gains of any gene in 4-gene FISH probe analysis. Using a threshold of >4 abnormal cells, inventor can predict cancer status in 21/23 cases with cancer; notice using this threshold, the inventor observed 100% specificity. X axis=number of patients, y axis=# of abnormal cells.

### DETAILED DESCRIPTION OF ILLUSTRATIVE EMBODIMENTS

Circulating tumor cells (CTCs) in patients with lung cancer will show genetic abnormalities similar to that seen in the primary lung cancer. Other investigators have used immunomagnetic capture or density gradient centrifugation with immunohistochemistry and FISH to detect aneuploidy in CTCs. However all studies, while demonstrating genetic abnormalities similar to those of the primary tumor, were limited by a low cell recovery and inability to detect chromosomal abnormalities in patients with CTCs<10 per 7.5 mL blood.

Genetically abnormal mononuclear cells (or circulating tumor cells) containing the same genetic abnormality as the primary tumor are present in peripheral blood of lung cancer patients, are associated with tumor stage and tumor burden, and occur at lower levels in patients with low stage versus high stage disease. Monitoring of these cells in the peripheral blood by combined immunocytochemistry and fluorescence in situ hybridization (FISH), at both at baseline and at follow up after therapy, provides a sensitive molecular marker of response to therapy if the number of cells bearing these chromosomal or genetic abnormalities decrease. In some cases, the inventor has shown that the level CTCs may peak 3-6 weeks post surgical removal of the lung cancer, and be far higher than the original blood sample taken at baseline before surgery. She ascribes this second peak to CTCs moving from sequestered sites such as the bone marrow into the peripheral blood, and a third follow up blood taken 12 weeks later, may fall back to below baseline levels. This finding is associated with longer survival. Similarly, persistence or increased numbers of cells with these deletions will indicate stable or progressive disease. For example, deletions in chromosome 3p21.3 and in 3p22.1 occur simultaneously, and very early on in the pathogenesis of early lung neoplasia. There are numerous tumor suppressor genes located in this portion of the genome that are highly relevant to lung cancer neoplasia (Barkan et al., 2004; Goeze et al., 2002). Similarly deletions on chromosome 10q22-23 have been frequently reported in primary lung cancer and also in metastatic lung cancer, both for small cell and non-small cell carcinoma (NSCL). Deletions of 10q22-23 furthermore are associated with an aggressive clinical course, with high levels of deletions being strongly associated with poor prognosis Jiang et al., 2005; Goeze et al., 2002; Gough et al., 2002).

The present disclosure therefore provides for methods of isolating the tumor cells from the peripheral blood using a nuclear size exclusion method, FISH analysis using validated nucleic acid probe sets, for the detection of cancer, follow-up after therapy and for longitudinal monitoring of disease status and response to different therapies. It has been shown that by the methods of the present disclosure, cells with clonal genetic abnormalities could be found in peripheral blood at much higher levels compared to previous methods.

This method has the benefits of (1) the ability to isolate much higher numbers of abnormal cells than had previously been described by other methods, thereby permitting use of a smaller sample volumes; (2) the ability to perform multicolor FISH using a variety of molecular DNA probes on a single specimen combined with immuno-fluorescence staining in order to obtain a phenotype of the CTCs and to demonstrate clonality; and (3) the ability to enrich for the abnormal phenotype by examining cells that have appropriate nuclear size. In comparison with other methods, significant improvement in terms of sensitivity and accuracy were achieved.

It should be noted that the methods described in this application are applicable for isolating circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc. In particular, it is possible for each set of tumors, to derive a set of genomic markers that are abnormal in a specific cancer subtype based on published genomic data or on genomic data generated by testing different tumors with comparative genomic hybridization (CGH) or single nucleotide polymorphisms (SNPS) and performing bioinformatics to determine over- or underexpression of different genes. Following the best choice of abnormal molecular regions to be tested, the optimal fluorescently labeled probes can be synthesized.

### I. Cancer

The present disclosure envisions the use of assays to detect cancer and predict its progression in conjunction with cancer therapies. In some cases, where patients are suspected to be at risk of cancer, prophylactic treatments may be employed. In other cancer subjects, diagnosis may permit permit early therapeutic intervention. In yet other situations, the result of the assays described herein may provide useful information regarding the need for repeated treatments, for example, where there is a likelihood of metastatic, recurrent or residual disease. Finally, the present disclosure may prove useful in demonstrating which therapies do and do not provide benefit to a particular patient.

Furthermore, the methods described in this application are able to be translated into a method for isolating circulating tumor cells from any other type of cancer that gives rise to blood borne metastases. This would include cancers of lung, breast, colon, prostate, pancreas, esophagus, all gastro-intestinal tumors, urogenital tumors, kidney cancers, melanomas, endocrine tumors, sarcomas, etc.

A. Tumorgenesis

The deletion of various genes in tumor tissue has been well studied in the art. However, there remains a need for probes that are significant for detecting early molecular events in the development of cancers, as well as molecular events that make patients susceptible to the development of cancer. Probes used for the staging of cancer are also of interest. The proposed sequence leading to tumorigenesis includes genetic instability at the cellular or submicroscopic level as demonstrated by loss or gain of chromosomes, leading to a hyperproliferative state due to theoretical acquisition of factors that confer a selective proliferative advantage. Further, at the genetic level, loss of function of cell cycle inhibitors and tumor suppressor genes (TSG), or amplification of oncogenes that drive cell proliferation, are implicated.

Following hyperplasia, a sequence of progressive degrees of dysplasia, carcinoma-in-situ and ultimately tumor invasion is recognized on histology. These histologic changes are both preceded and paralleled by a progressive accumulation of genetic damage. At the chromosomal level genetic instability is manifested by a loss or gain of chromosomes, as well as structural chromosomal changes such as translocation and inversions of chromosomes with evolution of marker chromosomes. In addition cells may undergo polyploidization. Single or multiple clones of neoplastic cells may evolve characterized in many cases by aneuploid cell populations. These can be quantitated by measuring the DNA content or ploidy relative to normal cells of the patient by techniques such as flow cytometry or image analysis.

B. Prognostic Factors and Staging

The stage of a cancer at diagnosis is an indication of how much the cancer is spread and can be one of the most important prognostic factors regarding patient survival. Staging systems are specific for each type of cancer. For example, at present the most important prognostic factor regarding the survival of patients with lung cancer of non-small cell type is the stage of disease at diagnosis. For example, the most important prognostic factor regarding the survival of patients with lung cancer of non-small cell type is the stage of disease at diagnosis. Conversely, small cell cancer usually presents with wide spread dissemination hence the staging system is less applicable. The staging system was devised based on the anatomic extent of cancer and is now know as the TNM (Tumor, Node, Metastasis) system based on anatomical size and spread within the lung and adjacent structures, regional lymph nodes and distant metastases. The only hope presently for a curative procedure lies in the operability of the tumor which can only be resected when the disease is at a low stage when confined to the organ of origination.

C. Grading of Tumors

The histological type and grade of lung cancers do have some prognostic impact within the stage of disease with the best prognosis being reported for stage I adenocarcinoma, with 5 year survival at 50% and 1-year survival at 65% and 59% for the bronchiolar-alveolar and papillary subtypes (Naruke et al., 1988; Travis et al., 1995; Carriaga et al., 1995). For squamous cell carcinoma and large cell carcinoma the 5 year survival is around 35%. Small cell cancer has the worst prognosis with a 5 year survival rate of only 12% for patients with localized disease (Carcy et al., 1980; Hirsh, 1983; Vallmer et al., 1985). For patients with distant metastases survival at 5 years is only 1-2% regardless of histological subtype (Naruke et al., 1988). In addition to histological subtype, it has been shown that histological grading of carcinomas within subtype is of prognostic value with well differentiated tumors having a longer overall survival than poorly differentiated neoplasms. Well differentiated localized adencarcinoma has a 69% overall survival compared to a survival rate of only 34% of patients with poorly differentiated adenocarcinoma (Hirsh, 1983). The 5 year survival rates of patients with localized squamous carcinoma have varied from 37% for well differentiated neoplasms to 25% for poorly differentiated squamous carcinomas (Ihde, 1991).

The histologic criteria for subtyping lung tumors are as follows: squamous cell carcinoma consists of a tumor with keratin formation, keratin pearl formation, and/or intercellular bridges. Adenocarcinomas consist of a tumor with definitive gland formation or mucin production in a solid tumor. Small cell carcinoma consists of a tumor composed of small cells with oval or fusiform nuclei, stippled chromatin, and indistinct nuclei. Large cell undifferentiated carcinoma consists of a tumor composed of large cells with vesicular nuclei and prominent nucleoli with no evidence of squamous or glandular differentiation. Poorly differentiated carcinoma includes tumors containing areas of both squamous and glandular differentiation.

D. Development of Carcinomas

The evolution of carcinoma of the lung is most likely representative of a field cancerization effect as a result of the entire aero-digestive system being subjected to a prolonged period of carcinogenic insults such as benzylpyrenes, asbestosis, air pollution and chemicals other carcinogenic substances in cigarette smoke or other environmental carcinogens. This concept was first proposed by Slaughter et al. (1953). Evidence for existence of a field effect is the common occurrence of multiple synchronous for metachronous second primary tumors (SPTs) that may develop throughout the aero-digestive tract in the oropharynx, upper esophagus or ipsilateral or contralateral lung.

Accompanying these molecular defects is the frequent manifestation of histologically abnormal epithelial changes including hyperplasia, metaplasia, dysplasia, and carcinoma-in-situ. It has been demonstrated in smokers that both the adjacent normal bronchial epithelium as well as the preneoplastic histological lesions may contain clones of genetically altered cells (Wistuba et al., 2000).

Licciardello et al. (1989) found a 10-40% incidence of metachronous tumors and a 9-14% incidence of synchronous SPTs in the upper and lower aero-digestive tract, mostly in patients with the earliest primary tumors SPTs may impose a higher risk than relapse from the original primary tumor and may prove to be the major threat to long term survival following successful therapy for early stage primary head, neck or lung tumors. Hence it is vitally important to follow these patients carefully for evidence of new SPTs in at risk sites for new malignancies specifically in the aero-digestive system.

In addition to chromosomal changes at the microscopic level, multiple blind bronchial biopsies may demonstrate various degrees of intraepithelial neoplasia at loci adjacent to the areas of lung cancer. Other investigators have shown that there are epithelial changes ranging from loss of cilia and basal cell hyperplasia to CIS in most light and heavy smokers and all lungs that have been surgically resected for cancer (Auerbach et al., 1961). Voravud et al. (1993) demonstrated by in-situ hybridization (ISH) studies using chromosome-specific probes for chromosomes 7 and 17 that 30-40% of histologically normal epithelium adjacent to tumor showed polysomies for these chromosomes. In addition there was a progressive increase in frequency of polysomies in the tissue closest to the carcinoma as compared to normal control oral epithelium from patients without evidence of carcinoma. The findings of genotypic abnormalities that increased closer to the area of the tumor support the concept of field cancerization. Interestingly, there was no increase in DNA content as measured in the normal appearing mucosa in a Feulgen stained section adjacent to the one where the chromosomes were measured, reflecting perhaps that insufficient DNA had been gained in order to alter the DNA index. Interestingly, a very similar increase in DNA content was noted both in dysplastic areas close to the cancer and in the cancerous areas suggesting that complex karyotypic abnormalities that are clonal have already been established in dysplastic epithelium adjacent to lung cancer. Others have also shown an increase in number of cells showing p53 mutations in dysplastic lesions closest to areas of cancer, which are invariably also p53 mutated. Other chromosomal abnormalities that have recently been demonstrated in tumors and dysplastic epithelium of smokers includes deletions of 3p, 17p, 9 p and 5q (Feder et al., 1998; Yanagisawa et al., 1996; Thiberville et al., 1995).

E Chromosome Deletions in Lung Cancer

Small cell lung cancer (SCLC) and non-small cell lung cancer commonly display cytogenetically visible deletions on the short arm of chromosome 3 (Hirano et al., 1994; Valdivieso et al., 1994; Cheon et al., 1993; Pence et al., 1993). This 3p deletion occurs more frequently in the lung tumor tissues of patients who smoke than it does in those of nonsmoking patient. (Rice et al., 1993) Since approximately 85% lung cancer patients were heavy cigarette smokers (Mrkve et al., 1993), ³p might contain specific DNA loci related to the exposure of tobacco carcinogens. It also has been reported that 3p deletion occurs in the early stages of lung carcinogenesis, such as bronchial dysplasia (Pantel et al., 1993). In addition to cytogenetic visible deletions, loss of heterozygosity (LOH) studies have defined 3-21.3 as one of the distinct regions that undergo loss either singly or in combination (Fontanini et al., 1992; Liewald et al., 1992). Several other groups have found large homozygous deletions at 3p21.3 in lung cancer (Macchiarini et al., 1992; Miyamoto et al., 1991; Ichinose et al., 1991; Yamaoka et al., 1990). Transfer of DNA fragments from 3-21.3-3p21.2 into lung tumor cell lines could suppress the tumorigenesis (Sahin et al., 1990; Volm et al., 1989). These finding strongly suggest the presence of at least one tumor suppressor gene in this specific chromosome region whose loss will initiate lung carcinogenesis.

Cytogenetic observation of lung cancer has shown an unusual consistency in the deletion rate of chromosome 3p. In fact, small cell lung cancer (SCLC) demonstrates a 100% deletion rate within certain regions of chromosome 3p. Non small cell lung cancer (NSCLC) demonstrates a 70% deletion rate (Mitsudomi et al., 1996; Shiseki et al., 1996). Loss of heterozygosity and comparative genomic hybridization analysis have shown deletions between 3p14.2 and 3p21.3 to be the most common finding for lung carcinoma and is postulated to be the most crucial change in lung tumorigenesis (Wu et al., 1998). It has been hypothesized that band 3p21.3 is the location for lung cancer tumor suppressor genes. The hypothesis is supported by chromosome 3 transfer studies, which reduced tumorigenicity in lung adenocarcinoma.

Allelotype studies on non-small cell lung carcinoma indicated loss of genetic material on chromosome 10q in 27% of cases. Studies of chromosome 10 allelic loss have shown that there is a very high incidence of LOH in small cell lung cancer, up to 91% (Alberola et al., 1995; Ayabe et al., 1994). A statistically significant LOH of alleles on 10q was noted in metastatic squamous cell carcinoma (SCC) in 56% of cases compared to non-metastatic SCC with LOH seen in only 14% of cases (Ayabe et al., 1994). No LOH was seen in other subtypes on NSCLC. Additionally, using micro-satellite polymorphism analysis, it was shown that a high incidence of loss exists between D10s677 and D10S1223. This region spans the long arm of chromosome 10 at bands q21-q24 and overlaps the region deleted in the a study of advanced stage high grade bladder cancers which demonstrated a high frequency of allele loss within a 2.5 cM region at 10q22.3-10q23.1 (Kim et al., 1996).

### II. Sorting and Selection by Nuclear Size

In one aspect, the disclosure provides for isolating and/or classifying CTCs according to nuclear size or nucleus/cytoplasm ratio. These methods may involve physical sorting, such as by FACS or other nuclei sorting means, but analysis of optical data using a computer-driven size analysis, or by manual interrogation of cell nuclei, such as by using standard light microscopy. Typically, the nuclei are stained in order to permit assessment/sorting, such as with DAPI (4′,6-diamidino-2-phenylindole). In certain embodiments, the nuclei will be obtained from cells and sorted on their own. Cells may be lysed using standard cells lysis protocols.

A. Bioview System and Software

The Bioview Duet™ (Rehovot, Israel) system use a color or monochromatic CCD cameras normally images and classifies all nucleated cells presented on the cytopreparation. The number of cells classified is preset by the operator however usually several thousand cells are scanned. There is a “research” mode or an open software system, that then records for each cell:
1) nuclear area in pixels, based on the DAPI stain, expressed as arbitrary units, thus, if 5000 it means that the cell area is 5000 pixels;
2) nuclear diameter; and
3) circularity factor (CFs), calculated by modifying the elongation (proportion between the height and the width of the cell) where a perfect circle will have the value of 1 (lymphocytes have CFs close to 1, abnormal cells have much CFs>>1, due to their nuclear perimeter irregularity).
   In order to increase the yield of CTCs, the inventor made the following measurements and then adjusted the software so as to enhance the yield of abnormal cells and decrease the numbers of normal lymphocytes.

The nuclear area for the abnormal (malignant CTCs) cells was based on the number of pixels occupied by the nucleus (as defined by FISH polysomy >2) as measured on the DAPI stain (a nuclear stain) and was expressed in arbitrary units.

The nuclear area for the lymphocytes was the number of pixels occupied by the lymphocytes in the blood that were were diploid by FISH, with a circularity factor close to 1. The way the measurement was derived was from observing the average nuclear pixel area of the lymphocytes from numerous malignant specimens (“internal” control lymphocytes) as well as recording the average nuclear pixel area of lymphocytes within control specimens or “external” control lymphocytes, from patients known to be healthy without history of prior malignancy or malignant cells in their blood streams. Similarly, observations were recorded of the nuclear area of numerous “abnormal” cells (circulating tumor cells) defined as cells with 2 or more polysomies (extra chromosomes) from patients with known lung cancer. The inventor showed that the nuclear areas for the CTCs far exceeded the arbitrary threshold, as discussed below.

In embodiments where absolute numbers of CTCs are diagnostic, a finding of 4 or more CTCs will indicate that the patient has cancer. The inventor notes that some patients in remission for several years can show a few CTCs (minimal residual disease; less than 4 CTCs as defined herein) which may represent dormant CTCs. The half life of CTCs is said to be about 4-8 hours, so there is a constantly replenishing source. This is currently a phenomenon of great biological relevance, as after several years of apparent “remission” patients can relapse and die, very likely implicating these dormant CTCs.

B. Threshold

A threshold of 78 was chosen based on the average pixel area of lymphocytes with a CF close to 1, within the blood from patients who had lung cancer. This threshold value was significantly lower than the average pixels noted for abnormal cells (defined by FISH polysomy >2).

C. Classification

A duplicate task with exclusions was created so that the system would only start classifying cells within the Ficoll purified specimen that were larger than 78. Thus, all cells less than 78, comprising the average nuclear area of lymphocytes were excluded, and only the cells that meet the derived criterion (threshold >78) were classified and presented to the operator for interactive evaluation. In addition, the Bioview system creates a pie chart to display dipoid cells, aneuploid cells (single gains or losses) and abnormal cells (at least polysomy of 2 or more genes as defined by FISH probes, 3cen, 3p, 10cen and 10q).

The instrument task is set to scan several thousand cells so that at least 500 intact and non-overlapped cells with the derived criterion (threshold >78) can be selected from several thousand images, which are presented to the operator for interactive evaluation of extra signals (gains) or loss of signals (deletions).

When evaluating the scanned cells, the operator will first check different categories of cells according to the pie chart, beginning with the “abnormal” cells which are defined as at least 2 chromosomes with extra copies, then the single gain and loss categories, and finally the remaining cells will be interactively analysed until 500 cells have been scored.

### III. Gene Probes

The present disclosure comprises contacting the selected cells with a labeled nucleic acid probe, and detecting hybridized cells by fluorescence in situ hybridization. These probes may be specific for any genetic marker that is most frequently amplified or deleted in CTCs. In particular, the probes may be a 3p22.1 probe, which is a nucleic acid probe targeting RPL14, CD39L3, PMGM, or GC20, combined with centromeric 3; a 10q22-23 probe (encompassing surfactant protein A1 and A2) combined with centromeric 10; or a PI3 kinase probe. Other genetic markers may include, but are not limited to, centromeric 3, 7, 17, 9p21, 5p15.2, EGFR, C-myc8q22, and 6p22-22. For a further discussion of gene probes see U.S. Publication No. 2007/0218480, herein incorporated by reference in its entirety.

A. 3p22.1 Probe

A 3p22.1 probe is a nucleic acid probe targeting RPL14, CD39L3, PMGM, or GC20, combined with centromeric 3. The human ribosomal L14 (RPL14) gene (GenBank Accession NM_003973), and the genes CD39L3 (GenBank Accession AAC39884 and AP039917), PMGM (GenBank Accession P15259 and J05073), and GC20 (GenBank Accession NM_005875) were isolated from a BAC (GenBank Accession AC104186, herein incorporated by reference) and located in the 3p22.1 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. It is understood that the polyalanine tract plays a key role in the nonsense-mediated mRNA decay pathway that rids cells aberrant proteins and transcripts. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. See also RP11-391M1/AC104186.

Genes with a regulatory function such as the RPL14 gene, along with the genes CD39L3, PMGM, and GC20 and analogs thereof, are good candidates for diagnosis of tumorigenic events. It has been postulated that functional changes of the RPL14 protein can occur via a DNA deletion mechanism of the trinucleotide repeat encoding for the protein. This deletion mechanism makes the RPL14 gene an attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

B. 10q22 Probe

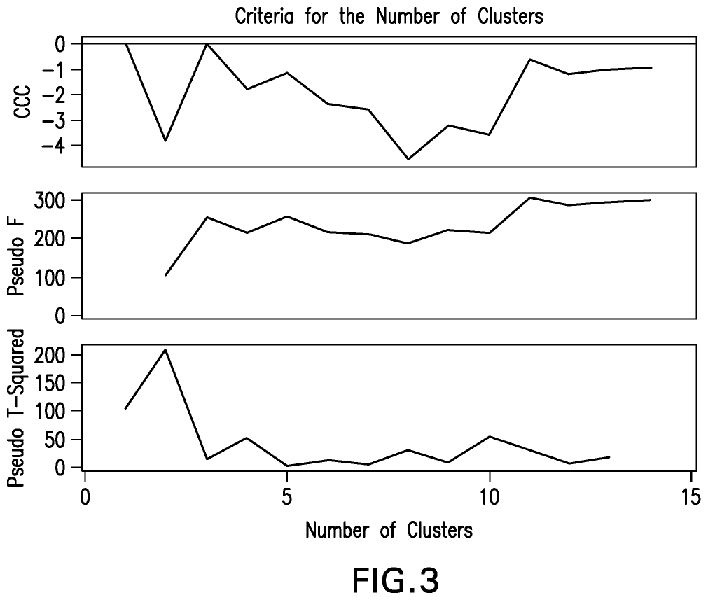

In other embodiments, the probe may be a 10q22-23 probe, which encompasses surfactant protein A1 and A2, combined with centromeric 10. The 10q22 BAC (46b12) is 200 Kb and is adjacent and centromeric to PTEN/MMAC1 (GenBank Accession AF067844), which is at 10q22-23 and can be purchased through Research Genetics (Huntsville, Ala.) (FIG. 3). Alterations to 10q22-25 has been associated with multiple tumors, including lung, prostate, renal, and endomentrial carcinomas, melanoma, and meningiomas, suggesting the possible suppressive locus affecting several cancers in this region. The PTEN/MMAC1 gene, encoding a dual-specificity phosphatase, is located in this region, and has been isolated as a tumor suppressor gene that is altered in several types of human tumors including brain, bladder, breast and prostate cancers. PTEN/MMAC1 mutations have been found in some cancer cell lines, xenografts, and hormone refractory cancer tissue specimens. Because the inventor's 10q22 BAC DNA sequence is adjacent to this region, the DNA sequences in the BAC 10q22 may be involved in the genesis and/or progression of human lung cancer. See also RP11-506M13/AC068139.6

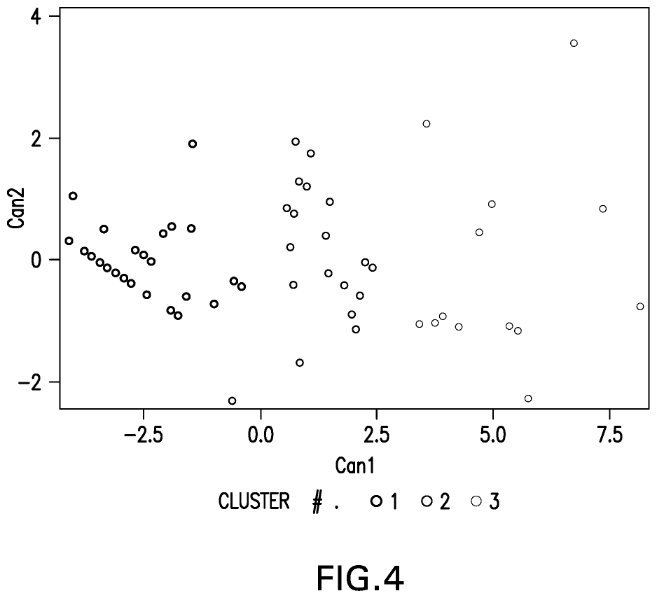

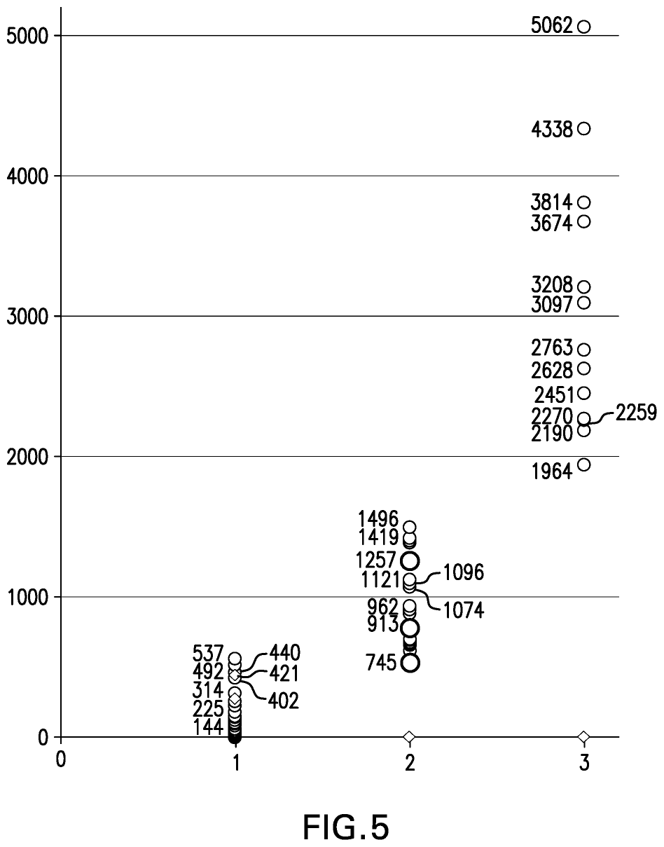

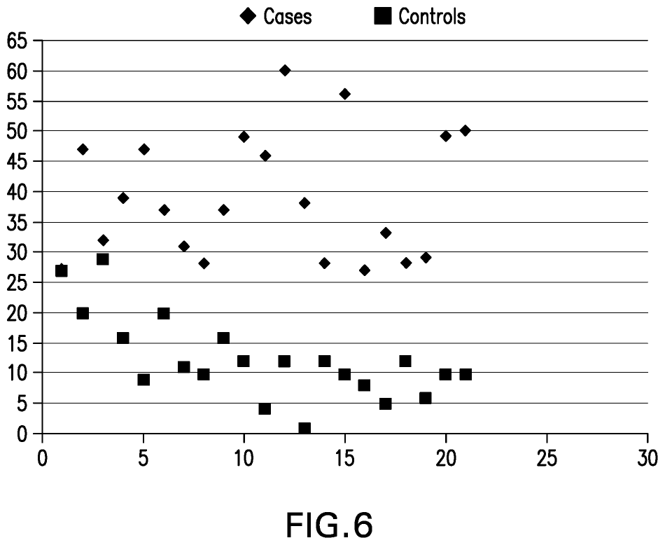

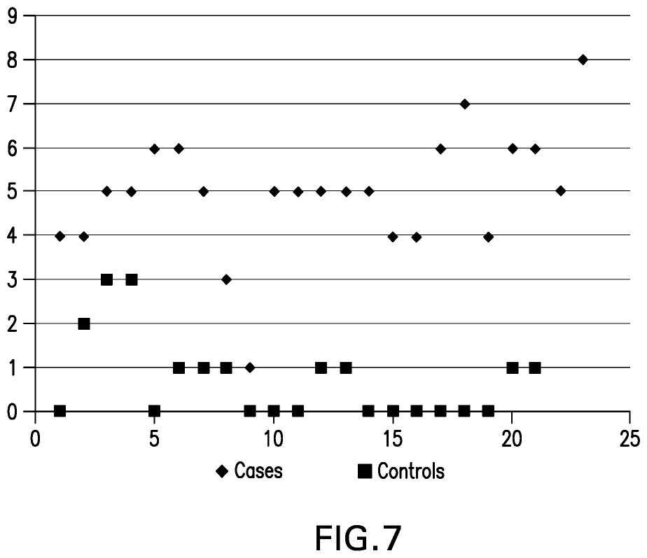

Pulmonary-associated surfactant protein A1 (SP-A) is located at 10q22.3. Surfactant protein-A-phospholipid-protein complex lowers the surface tension in the alveoli of the lung and plays a major role in host defense in the lung. Surfactant protein-A1 is also present in alveolar type-2 cells, which are believed to be putative stem cells of the lung. It is known that type-2 cells participate in repair and regeneration after alveolar damage. Thus, it is possible that the type-2 cells express telomerase and C-MYC, which leads to the loss of the surfactant protein and the development of non-small cell lung cancer (FIG. 4). The 10q22 probe is useful in the further development of clinical biomarkers for the early detection of neoplastic events, for risk assessment and monitoring the efficacy of chemoprevention therapy.

C. Commercial Probe Sets

Any commercial probes or probe sets may also be used with the present disclosure. For example, the UroVysion DNA probe set (Vysis/Abbott Molecular, Des Plaines, Ill.) may be used, which includes probes directed to centromeric 3, centromeric 7, centromeric 17, 9p21.3. It has been established that UroVysion probes detect early changes of lung cancer. In other embodiments, the LaVysion DNA probe set (Vysis/Abbott Molecular, Des Plaines, Ill.), which includes probes to 7p12 (epidermal growth factor receptor); 8q24.12-q24.13 (MYC); 6p11.1-q11 (chromosome enumeration (Probe CEP 6); and 5p15.2 (encompassing the SEMA5A gene), may be used. It has been noted that the LaVysion probe set detects higher stages or more advanced stags of lung cancer. Furthermore, a single probe set directed to centromeric7/7p12 (epidermal growth factor receptor) may also be used with the present disclosure.

### IV. Methods for Assessing Gene Structure

In accordance with the present disclosure, one will utilize various probes to examine the structure of genomic DNA from patient samples. A wide variety of methods may be employed to detect changes in the structure of various chromosomal regions. The following is a non-limiting discussion of such methods.

A. Fluorescence In Situ Hybridization and Chromogenic In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout its length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.). Next, the probe is placed on the slide and the slide is placed in a 37° C. incubator overnight for the probe to hybridize with the target chromosome. Overnight, the probe DNA seeks out its target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for loss of allelic heterozygosity is that the:
(a) FISH is easily and rapidly performed on cells of interest and can be used on paraffin-embedded, or fresh or frozen tissue allowing the use of micro-dissection;
(b) specific gene changes can be analyzed on a cell by cell basis in relationship to centomeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted);
(c) PCR cannot identify amplification of genes; and
(d) FISH using bacterial artificial chromosomes (BACs) permits easy detection and localization on specific chromosomes of genes of interest which have been isolated using specific primer pairs.

Chromogenic in situ hybridzation (CISH) enables the gain genetic information in the context of tissue morphology using methods already present in histology labs. CISH allows detection of gene amplification, chromosome translocations and chromosome number using conventional enzymatic reactions under the brightfield microscope on formalin-fixed, paraffin-embedded (FFPE) tissues. U.S. Publication No. 2009/0137412, incorporated herein by reference. The scanning may be performed, for example, on an automated scanner with Fluorescence capabilities (Bioview System, Rehovot, Israel).

B. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA.RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure (Wu et al., 1989, incorporated herein by reference in its entirety).

C. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

D. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present disclosure: adsorption, partition, ion-exchange and molecular sieve, and many specialized techniques for using them including column, paper, thin-layer and gas chromatography (Freifelder, 1982).

E Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al., 1994). The present disclosure provides methods by which any or all of these types of analyses may be used.

F. Kit Components

All the essential materials and reagents required for detecting changes in the chromosomal regions discussed above may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification, and optionally labeling agents such as those used in FISH. Such kits also generally will comprise, in suitable means, distinct containers for each individual reagent and enzyme as well as for each primer or probe.

G. Chip Technologies

Specifically contemplated by the present inventors are chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules using methods such as fluorescence, conductance, mass spectrometry, radiolabeling, optical scanning, or electrophoresis. See also Pease et al. (1994); Fodor et al. (1991).

Biologically active DNA probes may be directly or indirectly immobilized onto a surface to ensure optimal contact and maximum detection. When immobilized onto a substrate, the gene probes are stabilized and therefore may be used repetitively. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Immobilization of the gene probes may be achieved by a variety of methods involving either non-covalent or covalent interactions between the immobilized DNA comprising an anchorable moiety and an anchor. DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. Gene probe may be bound directly to membranes using ultraviolet radiation. With nitrocellous membranes, the probes are spotted onto the membranes. A UV light source is used to irradiate the spots and induce cross-linking. An alternative method for cross-linking involves baking the spotted membranes at 80° C. for two hours in vacuum.

Immobilization can consist of the non-covalent coating of a solid phase with streptavidin or avidin and the subsequent immobilization of a biotinylated polynucleotide (Holmstrom, 1993). Precoating a polystyrene or glass solid phase with poly-L-Lys or poly L-Lys, Phe, followed by the covalent attachment of either amino- or sulfhydryl-modified polynucleotides using bifunctional crosslinking reagents (Running, 1990; Newton, 1993) can also be used to immobilize the probe onto a surface.

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287) describes a method of non-covalently immobilizing nucleic acid molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the synthetic nucleic acid and the cationic detergent or salt. The support containing the immobilized nucleic acid may be washed with an aqueous solution containing a non-ionic detergent without removing the attached molecules.

There are two common variants of chip-based DNA technologies involving DNA microarrays with known sequence identity. For one, a probe cDNA (500-5,000 bases long) is immobilized to a solid surface such as glass using robot spotting and exposed to a set of targets either separately or in a mixture. This method, “traditionally” called DNA microarray, is widely considered as developed at Stanford University. A recent article by Ekins and Chu (1999) provides some relevant details. The other variant includes an array of oligonucleotide (20-25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its products under the GeneChip® trademark.

### V. Nucleic Acids

The inventors provides a method comprises a step of contacting the selected cells with a labeled nucleic acid probe forming hybridized cells, wherein hybridization of the labeled nucleic acid is indicative of a CTC. However, the present disclosure is not limited to the use of the specific nucleic acid segments disclosed herein. Rather, a variety of alternative probes that target the same regions/polymorphisms may be employed.

A. Probes and Primers

Naturally, the present disclosure encompasses DNA segments that are complementary, or essentially complementary, to target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a target nucleic acid segment under relatively stringent conditions such as those described herein. These probes may span hundreds or thousands of base pairs.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of about 8, 9, 10, 11, 12, 13, 14, 15, 16, 17, 18, 19, 20, 25, 30, 35, 40, 45, 50, 55, 60, 65, 70, 75, 80, 85, 90, 95, 100, 250, 500, 700, 722, 900, 992, 1000, 1500, 2000, 2500, 2800, 3000, 3500, 3800, 4000, 5000 or more base pairs will be used, although others are contemplated. As mentioned above, longer polynucleotides encoding 10,000, 50,000, 100,000, 150,000, 200,000, 250,000, 300,000 and 500,000 bases are contemplated. Such oligonucleotides and polynucleotides will find use, for example, as probes in FISH, Southern and Northern blots and as primers in amplification reactions.

It will be understood that this disclosure is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present disclosure are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

B. Labeling of Probes

In certain embodiments, it will be advantageous to employ nucleic acid sequences of the present disclosure in combination with an appropriate means, such as a label, for determining hybridization. A wide variety of appropriate indicator means are known in the art, including fluorescent, radioactive, chemiluminescent, electroluminescent, enzymatic tag or other ligands, such as avidin/biotin, antibodies, affinity labels, etc., which are capable of being detected. In preferred embodiments, one may desire to employ a fluorescent label such as digoxigenin, spectrum orange, fluorosein, eosin, an acridine dye, a rhodamine, Alexa 350, Alexa 430, AMCA, BODIPY 630/650, BODIPY 650/665, BODIPY-FL, BODIPY-R6G, BODIPY-TMR, BODIPY-TRX, cascade blue, Cy2, Cy3, Cy5,6-FAM, HEX, 6-JOE, Oregon green 488, Oregon green 500, Oregon green 514, pacific blue, REG, ROX, TAMRA, TET, or Texas red.

In the case of enzyme tags such as urease alkaline phosphatase or peroxidase, colorimetric indicator substrates are known which can be employed to provide a detection means visible to the human eye or spectrophotometrically, to identify specific hybridization with complementary nucleic acid-containing samples. Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

The indicator means may be attached directly to the probe, or it may be attached through antigen bonding. In preferred embodiments, digoxigenin is attached to the probe before denaturization and a fluorophore labeled anti-digoxigenin FAB fragment is added after hybridization.

C. Hybridization Conditions

Suitable hybridization conditions will be well known to those of skill in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

### VI. Biomarkers and Other Risk Factors

Various biomarkers of prognostic significance can be used in conjunction with the specific nucleic acid probes discussed above. These biomarkers could aid in predicting the survival in low stage cancers and the progression from preneoplastic lesions to invasive lung cancer. These markers can include proliferation activity as measured by Ki-67 (MIB 1), angiogenesis as quantitated by expression of VEGF and microvessels using CD34, oncogene expression as measured by erb B2, and loss of tumor suppresser genes as measured by p53 expression.

Multiple biomarker candidates have been implicated in the evolution of neoplastic lung lesions. Bio-markers that have been studies include general genomic markers including chromosomal alterations, specific genomic markers such as alterations in proto-oncogenes such as K-Ras, Erb$1/EGFR, Cyclin D; proliferation markers such as Ki67 or PCNA, squamous differentiation markers, and nuclear retinoid receptors (Papadimitrakopoulou et al., 1996) The latter are particularly interesting as they may be modulated by specific chemopreventive drugs such as 13-cis-retinoic acid or 4HPR and culminate in apoptosis of the defective cells with restoration of a normally differentiated mucosa (Zou et al., 1998).

A. Tumor Angiogenesis by Microvessel Counts

Tumor angiogenesis can be quantitated by microvessel density and is a viable prognostic factor in stage 1 NSCLC. Tumor microvessel density appears to be a good predictor of survival in stage 1 NSCLC.

B. Vascular Endothelial Growth Factor (VEGF)

VEGF (3,6-8 ch 4) an endothelial cell specific mitogen is an important regulator of tumor angiogenesis who's expression correlates well with lymph node metastases and is a good indirect indicator of tumor agniogenesis. VEGF in turn is upregulated by P53 protein accumulation in NSCLC.

C. p53

The role of p53 mutations in predicting progression and survival of patients with NSCLC is widely debated. Although few studies imply a negligible role, the majority of the studies provide compelling evidence regarding the role of p53 as one of the prognostic factors in NSCLC. The important role of p53 in the biology of NSCLC has been the basis for adenovirus mediated p53 gene transfer in patients with advanced NSCLC (Carcy et al., 1980). In addition p53 has also been shown to be an independent predictor of chemotherapy response in NSCLC. In a recent study (Vallmer et al., 1985), the importance of p53 accumulation in preinvasive bronchial lesions from patients with lung cancer and those who did not progress to cancer were studied. It was demonstrated that p53 accumulation in preneoplastic lesions had a higher rate of progression to invasion than did p53 negative lesions.

D. c-erb-B2

Similar to p53, c-erg-B2 (Her2/neu) expression has also been shown to be a good marker of metastatic propensity and an indicator of survival in these tumors.

E. Ki-67 Proliferation Marker

In addition to the above markers, tumor proliferation index as measured by the extent of labeling of tumor cells for Ki-67, a nuclear antigen expressed throughout cell cycle correlates significantly with clinical outcome in Stage 1 NSCLC (Feinstein et al., 1970). The higher the tumor proliferation index the poorer is the disease free survival labeling indices provides significant complementary, if not independent prognostic information in Stage 1 NSCLC, and helps in the identification of a subset of patients with Stage 1 NSCLC who may need more aggressive therapy.

Alterations in the 3p21.3 and 10q22 loci are known to be associated with a number of cancers. More specifically, point mutations, deletions, insertions or regulatory perturbations relating to the 3p21.3 and 10q22 loci may cause cancer or promote cancer development, cause or promoter tumor progression at a primary site, and/or cause or promote metastasis. Other phenomena at the 3p21.3 and 10q22 loci include angiogenesis and tissue invasion. Thus, the present inventors have demonstrated that deletions at 3p21.3 and 10q22 can be used not only as a diagnostic or prognostic indicator of cancer, but to predict specific events in cancer development, progression and therapy.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited.

F. Surfactant Protein A and B

There are four main surfactant proteins: SP-A, B, C, and D. SP-A and D are hydrophilic, while SP-B and C are hydrophobic. The proteins are very sensitive to experimental conditions (temperature, pH, concentration, substances such as calcium, and so on). Moreover, their effects tend to overlap and thus it is difficult to pinpoint the specific role of each protein.

1. SP-A

SP-A was the first surfactant protein to be identified, and is also the most abundant (Ingenito et al., 1999). Its molecular mass varies from 26-38 kDa (Prez-Gil et al., 1998). The protein has a “bouquet” structure of six trimers (Haagsman and Diemel, 2001), and can be found in an open or closed form depending on the other substances present in the system. Calcium ions produce the closed-bouquet form (Palaniyar et al., 1998).

SP-A plays a role in immune defense. It is also involved in surfactant transport/adsorption (with other proteins). SP-A is necessary for the production of tubular myelin, a lipid transport structure unique to the lungs. Tubular myelin consists of square tubes of lipid lined with protein (Palaniyar et al., 2001). Mice genetically engineered to lack SP-A have normal lung structure and surfactant function, and it is possible that SP-A's beneficial surfactant properties are only evident under situations of stress (Korfhagen et al., 1996).

2. SP-B

Papillary thyroid carcinoma (PTC) is clinically heterogeneous. Apart from an association with ionizing radiation, the etiology and molecular biology of PTC is poorly understood. Using oligo-based DNA arrays to study expression profiles of eight matched pairs of normal thyroid and PTC tissues, Immunohistochemical analysis detected SFTPB in 39/52 ITCs, but not in follicular thyroid carcinoma and normal thyroid tissue. Huang et al. (2001.

G. Patient Interview and Other Risk Factors

In addition to analyzing the presence or absence of polymorphisms, as discussed above, it may be desirable to evaluate additional factors in a patient. For example, a patient interview, which would include a smoking history (years smoking, pack/day, etc.) is highly relevant to the diagnosis/prognosis. Also, the presence or absence of morphologic changes in sputum cells (squamous metaplasia, dysplasia, etc.) and a genetic instability score (genetic instability=composing the sum of abnormalities from various combinations in epithelial and neutrophils in sputum and/or peripheral blood cells or bone marrow cells or stem cells isolated from blood or bone marrow) may be used.

### VII. Obtaining and Purifying Samples

In accordance with the present disclosure, one will obtain a biological sample that contains blood cells. In some embodiments, the entity evaluating the sample for CTC levels did not directly obtain the sample from the patient. Therefore, methods of the disclosure involve obtaining the sample indirectly or directly from the patient. To achieve these methods, a doctor, medical practitioner, or their staff may obtain a biological sample for evaluation. The sample may be analyzed by the practitioner or their staff, or it may be sent to an outside or independent laboratory. The medical practitioner may be cognizant of whether the test is providing information regarding a quantitative level of CTCs.

In any of these circumstances, the medical practitioner may know the relevant information that will allow him or her to determine whether the patient can be diagnosed as having an aggressive form of cancer and/or a poor cancer prognosis based on the level of CTCs. It is contemplated that, for example, a laboratory conducts the test to determine the level of CTCs. Laboratory personnel may report back to the practitioner with the specific result of the test performed.

Typically, the sample is isolated from a biological sample taken from the individual, such as a blood sample or tissue sample using standard techniques such as disclosed in Jones (1963) which is hereby incorporated by reference. Collection of the samples may be by any suitable method, although in some aspects collection is by needle, catheter, syringe, scrapings, and so forth.

The sample may be prepared in any manner known to those of skill in the art. For example, the circulating epithelial cells from peripheral blood may be isolated from buffy layer following Ficoll-Hypaque gradient separation, allowing for enrichment of mononuclear cells (lymphocytes and epithelial cells). Other methods known to those of skill in the art may also be used to prepare the sample.

Nucleic acids may be isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. Depending on the format, the specific nucleic acid of interest is identified in the sample directly using amplification or with a second, known nucleic acid following amplification.

Following detection, one may compare the results seen in a given sample with a statistically significant reference group of samples from normal patients and patients that have or lack alterations in the various chromosome loci and control regions. In this way, one then correlates the amount or kind of alterations detected with various clinical states and treatment options.

### VIII. Cancer Treatments

In some embodiments, the disclosure provides compositions and methods for the diagnosis and treatment of breast cancer. In one embodiment, the disclosure provides a method of determining the treatment of cancer based on whether the level of CTCs is high in comparison to a control. The treatment may be a conventional cancer treatment. One of skill in the art will be aware of many treatments that may be combined with the methods of the present disclosure, some but not all of which are described below.

A. Formulations and Routes for Administration to Patients

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present disclosure administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the disclosure, methods include identifying a patient in need of treatment. A patient may be identified, for example, based on taking a patient history or based on findings on clinical examination.

B. Treatments

In some embodiments, the method further comprises treating a patient with breast cancer with a conventional cancer treatment. One goal of current cancer research is to find ways to improve the efficacy of chemo- and radiotherapy, such as by combining traditional therapies with other anti-cancer treatments. In the context of the present disclosure, it is contemplated that this treatment could be, but is not limited to, chemotherapeutic, radiation, a polypeptide inducer of apoptosis, a novel targeted therapy such as a tyrosine kinase inhibitor, or an anti-VEGF antibody, or other therapeutic intervention. It also is conceivable that more than one administration of the treatment will be desired.

1. Chemotherapy

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnnesyl-protein tansferase inhibitors, transplatinum, 5-fluorouracil, vincristin, vinblastin and methotrexate and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY 117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor, vaccines such as gene therapy vaccines and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of y-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as a H2A.Z targeting agent is administered. Delivery of a H2A.Z targeting agent in conjunction with a vector encoding one of the following gene products may have a combined anti-hyperproliferative effect on target tissues. A variety of proteins are encompassed within the disclosure, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present disclosure, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19 p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{NK}4 function by transfection with a plasmid expression vector reduced colony formation by some human cancer cell lines (Okamoto, 1994; Arap, 1995).

Other genes that may be employed according to the present disclosure include Rb, APC, DCC, NF-1, NF-2, WT-1, MEN-I, MEN-II, zac1, p73, VHL, MMAC1/H2A.Z, DBCCR-1, FCC, rsk-3, p27, p27/p16 fusions, p2l/p27 fusions, anti-thrombotic genes (e.g., COX-1, TFPI), PGS, Dp, E2F, ras, myc, neu, raf; erb, fms, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and the ICE-like proteases have both been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

d. RNA Interference (RNAI)

In certain embodiments, the H2A.Z inhibitor is a double-stranded RNA (dsRNA) directed to an mRNA for H2A.Z.

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). It is generally accepted that RNAi acts post-transcriptionally, targeting RNA transcripts for degradation. It appears that both nuclear and cytoplasmic RNA can be targeted (Bosher and Labouesse, 2000).

e. sIRNA

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998). siRNA are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

f. Production of Inhibitory Nucleic Acids

dsRNA can be synthesized using well-described methods (Fire et al., 1998). Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. As with the construction of DNA template libraries, a procedures may be used to aid this time intensive procedure. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25-mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells (Brummelkamp et al., 2002; Lee et al., 2002; Paul et al., 2002; Sui et al., 2002; Yu et al., 2002). Some of these plasmids are engineered to express shRNAs lacking poly (A) tails (Brummelkamp et al., 2002; Paul et al., 2002; Yu et al., 2002). Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ˜21 nt siRNA-like molecules (Brummelkamp et al., 2002). The siRNA-like molecules can, in turn, bring about gene-specific silencing in the transfected mammalian cells. g. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ, IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/RAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyperproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present disclosure or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

5. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### IX. Examples

The following examples are included to demonstrate certain non-limiting aspects of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Data. There are total 74 samples, 35 cases (27 unique individual case) and 39 (31 unique individual controls) controls. Of the 39 controls, 11 were newly added younger controls. The younger controls were different than Randa's controls, therefore, these controls were excluded from the analyses.

Of the remaining 63 samples (35 cases and 28 controls), there are 15 pairs, “C” with “F” (8 pairs being cases and 7 controls). The data of cases and controls were split into two sets according to “C” and “F” labeling. Each set contains 27 cases and 21 controls. Of the 8 paired cases, 7 belonged to “early stage” and 1 “late stage”. This “late staged” sample belonged to “Sarcomatoid” under histology and thus was not included in “Squamous cases vs Controls” analyses. Of the 7 “early staged” samples, 6 had “adeno” and 1 “squamous”.

TABLE 1
Fifteen pairs of samples containing “C” or “F” labeling

| Sample IDs | Case/Controls | Early/Late stage | Histology |
|---|---|---|---|
| VS13-107C | Case | Early | Squamous |
| VS13-107F | | Early | Squamous |
| VS13-108C | Case | Early | Adeno |
| VS13-108F | | Early | Adeno |
| VS13-109C | Case | Early | Adeno |
| VS13-109F | | | |
| VS13-111C | Control | | |
| VS13-111F | | | |
| VS13-116C | Case | Late | Sarcomatoid |
| VS13-116F | | Late | |
| VS13-125C | Case | Early | Adeno |
| VS13-125F | | Early | Adeno |
| VS13-126C | Case | Early | Adeno |
| VS13-126F | | Early | Adeno |
| VS13-127C | Control | | |
| VS13-127F | | | |
| VS13-131C | Case | Early | Adeno |
| VS13-131F | | Early | Adeno |
| VS13-134C | Control | | |
| VS13-134F | | | |
| VS13-136C | Control | | |
| VS13-136F | | | |
| VS13-142C | Control | | |
| VS13-142F | | | |
| VS13-144C | Control | | |
| VS13-144F | | | |
| VS13-145C | Control | | |
| VS13-145F | | | |
| VS13-148C | Case | Early | Adeno |
| VS13-148F | | Early | Adeno |

*Note:
this pair of samples were treated as “missing” or not included in the “Squamous cases vs Controls” analyses.

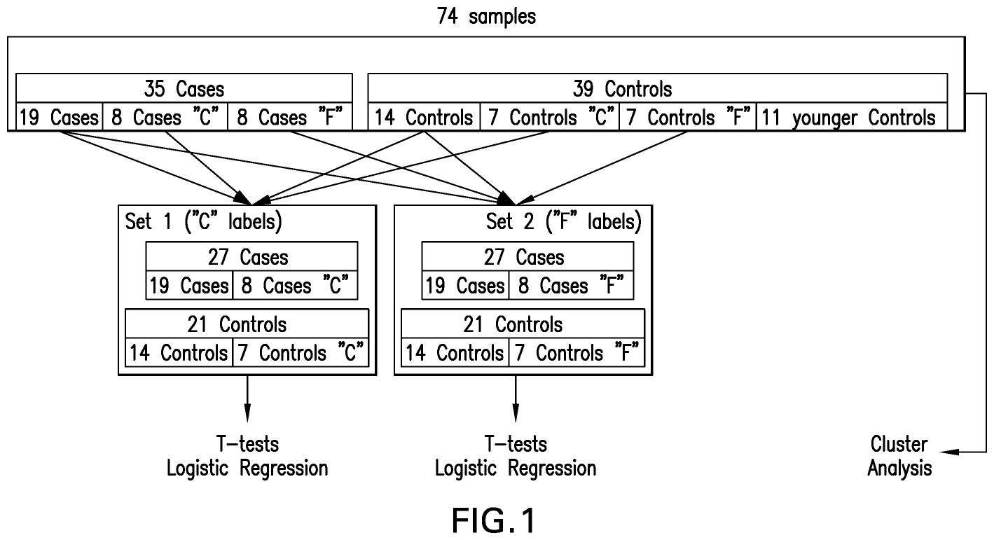

The data were grouped into two sets and each set contained 19 common cases and 14 common controls plus 8 cases and 7 controls with “C” label for set 1 or 8 cases and 7 controls with “F” label for set 2. See FIG. 1.

Variables. Three independent variables were examined, coded v1 for variable “# Of Abnormal Cells (>2 abnormalities)”, v2 for variable “# Of Total Abnormalities (dels+gains+abns),” and v3 for variable “# Of Abnormal Cells with Gains Only.”

Statistical Analyses. T-tests including nonparametric method using Wilcoxon and Kruskal-Wallis tests, univariate exact logistic regression and multivariate exact logistic regression (all three variables) over the cases and controls (overall, early staged with controls, late staged with controls, adeno with controls, and squamous with controls) were performed.

TABLE 2
Descriptive Statistics of Cases and Controls in Different Groups

| | | containing “C” labels | | | | | containing “F” labels | | | | |
|---|---|---|---|---|---|---|---|---|---|---|---|
| Groups | Variable | N | Mean | SD | min | max | N | Mean | SD | min | max |
| controls | stage | 21 | 0.00 | 0.00 | 0 | 0 | 21 | 0.00 | 0.00 | 0 | 0 |
| | hist | 21 | 0.00 | 0.00 | 0 | 0 | 21 | 0.00 | 0.00 | 0 | 0 |
| | v1 | 21 | 1.10 | 1.70 | 0 | 6 | 21 | 1.33 | 1.77 | 0 | 6 |
| | v2 | 21 | 12.48 | 7.02 | 1 | 29 | 21 | 14.00 | 7.87 | 1 | 35 |
| | v3 | 21 | 0.71 | 0.96 | 0 | 3 | 20 | 1.00 | 1.17 | 0 | 4 |
| Overall | stage | 27 | 1.11 | 0.32 | 1 | 2 | 27 | 1.11 | 0.32 | 1 | 2 |
| cases | hist | 24 | 1.42 | 0.50 | 1 | 2 | 24 | 1.42 | 0.50 | 1 | 2 |
| | v1 | 27 | 5.30 | 1.68 | 1 | 9 | 27 | 6.26 | 2.82 | 1 | 15 |
| | v2 | 27 | 36.07 | 10.11 | 18 | 60 | 27 | 38.04 | 13.71 | 15 | 70 |
| | v3 | 27 | 4.85 | 1.41 | 1 | 8 | 27 | 5.93 | 2.83 | 1 | 15 |
| Early | v1 | 24 | 5.17 | 1.71 | 1 | 9 | 24 | 6.25 | 2.98 | 1 | 15 |
| Stage | v2 | 24 | 34.58 | 9.16 | 18 | 56 | 24 | 38.17 | 14.30 | 15 | 70 |
| cases | v3 | 24 | 4.83 | 1.49 | 1 | 8 | 24 | 5.96 | 2.99 | 1 | 15 |
| Late | v1 | 3 | 6.33 | 1.15 | 5 | 7 | 3 | 6.33 | 1.15 | 5 | 7 |
| Stage | v2 | 3 | 48.00 | 11.14 | 38 | 60 | 3 | 37.00 | 9.54 | 27 | 46 |
| cases | v3 | 3 | 5.00 | 0.00 | 5 | 5 | 3 | 5.67 | 1.15 | 5 | 7 |
| cases of | v1 | 14 | 5.50 | 1.65 | 3 | 9 | 14 | 7.00 | 3.31 | 4 | 15 |
| histology | v2 | 14 | 36.50 | 10.61 | 18 | 56 | 14 | 41.00 | 17.08 | 15 | 70 |
| Adeno | v3 | 14 | 5.07 | 1.33 | 3 | 8 | 14 | 6.64 | 3.34 | 4 | 15 |
| cases of | v1 | 10 | 4.80 | 1.87 | 1 | 7 | 10 | 5.30 | 2.26 | 1 | 9 |
| histology | v2 | 10 | 32.80 | 7.51 | 27 | 47 | 10 | 35.10 | 9.21 | 27 | 51 |
| Squamous | v3 | 10 | 4.60 | 1.71 | 1 | 7 | 10 | 5.10 | 2.18 | 1 | 9 |

Note:
In “Stage”, 0 = control, 1 = early, and 2 = late.
In histology, 0 = control, 1 = adeno, and 2 = squamous, and anything else as missing.

### Example 2—Results

Cases are significantly different from controls over the three variables (v1, v2 and v3). Results of t-tests are all showing significant difference between cases and controls, no matter whether using equal-variance (when F-test is not significant) or unequal-variance method, or using non-parametric methods such as Wilcoxon test and Kruskal-Wallis test. This is the same in the set with “C” labeled samples as in the set with “F” ones. However, the major difference is seen in the variance test, i.e., F-test, between cases and controls in the set with “F” labels (bottom part of Table 3 below). This may indicate a greater heterogeneity between cases and controls containing the “F” samples.

TABLE 3
Results of F-test and t-tests
Samples containing “C”

| | | | t-test | | Wilcoxon test | | Kruskal- |
|---|---|---|---|---|---|---|---|
| | | | (equal | (unequal | Normal | t | Wallis test |
| | Variable | F-test | variance) | variance) | Approx | Approx | Chi-square |
| All Cases | v1 | 0.9469 | <0.0001 | <0.0001 | <0.0001 | <0.0001 | <0.0001 |
| vs Controls | v2 | 0.098 | <0.0001 | <0.0001 | <0.0001 | <0.0001 | <0.0001 |
| | v3 | 0.0809 | <0.0001 | <0.0001 | <0.0001 | <0.0001 | <0.0001 |
| Early Stage | v1 | 0.9848 | <0.0001 | <0.0001 | <0.0001 | <0.0001 | <0.0001 |
| vs Controls | v2 | 0.2329 | <0.0001 | <0.0001 | <0.0001 | <0.0001 | <0.0001 |
| | v3 | 0.0479 | <0.0001 | <0.0001 | <0.0001 | <0.0001 | <0.0001 |
| Late Stage | v1 | 0.726 | <0.0001 | 0.0042 | 0.0055 | 0.0107 | 0.0047 |
| vs Controls | v2 | 0.2118 | <0.0001 | 0.0258 | 0.0064 | 0.0121 | 0.0056 |
| | v3 | <0.0001 | <0.0001 | <0.0001 | 0.0039 | 0.0083 | 0.0033 |
| Adeno | v1 | 0.9403 | <0.0001 | <0.0001 | <0.0001 | <0.0001 | <0.0001 |
| vs Controls | v2 | 0.0937 | <0.0001 | <0.0001 | <0.0001 | <0.0001 | <0.0001 |
| | v3 | 0.1806 | <0.0001 | <0.0001 | <0.0001 | <0.0001 | <0.0001 |
| Squamous | v1 | 0.6797 | <0.0001 | <0.0001 | 0.0002 | 0.0007 | 0.0001 |
| vs Controls | v2 | 0.7577 | <0.0001 | <0.0001 | <0.0001 | 0.0002 | <0.0001 |
| | v3 | 0.0286 | <0.0001 | <0.0001 | <0.0001 | 0.0002 | <0.0001 |

Samples containing “F”

| | | | | | | | |
|---|---|---|---|---|---|---|---|
| All Cases | v1 | 0.0358 | <0.0001 | <0.0001 | <0.0001 | <0.0001 | <0.0001 |
| vs Controls | v2 | 0.0132 | <0.0001 | <0.0001 | <0.0001 | <0.0001 | <0.0001 |
| | v3 | 0.0002 | <0.0001 | <0.0001 | <0.0001 | <0.0001 | <0.0001 |
| Early Stage | v1 | 0.0216 | <0.0001 | <0.0001 | <0.0001 | <0.0001 | <0.0001 |
| vs Controls | v2 | 0.009 | <0.0001 | 0.0002 | <0.0001 | <0.0001 | <0.0001 |
| | v3 | 0.0001 | <0.0001 | <0.0001 | <0.0001 | 0.0002 | <0.0001 |
| Late Stage | v1 | 0.6816 | 0.0001 | 0.0044 | 0.0063 | 0.0119 | 0.0055 |
| vs Controls | v2 | 0.5074 | 0.0001 | 0.0421 | 0.0097 | 0.0165 | 0.0085 |
| | v3 | 1 | <.0001 | 0.0106 | 0.0051 | 0.0104 | 0.0044 |
| Adeno | v1 | 0.0122 | <0.0001 | <0.0001 | <0.0001 | <0.0001 | <0.0001 |
| vs Controls | v2 | 0.002 | <0.0001 | <0.0001 | <0.0001 | <0.0001 | <0.0001 |
| | v3 | <.0001 | <0.0001 | 0.0001 | <0.0001 | <0.0001 | <0.0001 |
| Squamous | v1 | 0.3444 | <.0001 | 0.0002 | 0.003 | 0.0059 | 0.0028 |
| vs Controls | v2 | 0.5312 | <0.0001 | <0.0001 | 0.0004 | 0.0013 | 0.0003 |
| | v3 | 0.0211 | <0.0001 | 0.0001 | 0.001 | 0.0025 | 0.0009 |

A significant result in F-test indicates the inequality of variance detected between cases and controls. Accordingly the t-test of unequal variance should be considered. The set containing “F” labeled samples, 8 cases and 7 controls, shows the strong tendency in variance difference between cases and controls. Referring to Table 1 above, such an effect of difference seems mainly coming from samples belonging to “early stage” and “adeno” groups. The results in “late stage” group may not be reliable due to extremely small sample size, i.e., only 3 cases, see Table 2 above.

Exact Logistic Regressions. Univariate exact logistic regression was carried out using cases of the five groups versus controls. Results show statistical significance across all groups in both sets containing “C” labels or “F” labels. The odds ratios (OR) are all greater than 1, implying a risk factor in all the three variables. Comparing the OR scale between the two sets, i.e. “C” and “F” sets, they are similar in each group, e.g. in the group “All Cases vs Controls”, OR=2.8 (v1 containing “C”) vs OR=2.7 (v1 containing “F”), 1.4 (v2 containing “C”) vs 1.26 (v2 containing “F”), and 5.78 (v3 containing “C”) vs 4.7 (v3 containing “F”). The failure to obtain the maximum likelihood estimation during exact logistic regression with variables v3 for the groups of “late stage cases vs controls” and “adeno cases vs controls” in both sets containing “C” labels or “F” labels (v2 as well in “C” labels) caused no reliable results achieved.

TABLE 4
Results of Exact Logistic Regression (univariate)
Samples containing “C”

| | | Beta | Standard | | | | | | | |
|---|---|---|---|---|---|---|---|---|---|---|
| Group | Variable | Estimate | Error | 95% | | p-value | OR | 95% | | p-value |
| All Cases | v1 | 1.0304 | 0.2637 | 0.5644 | 1.6945 | <.0001 | 2.8020 | 1.7580 | 5.4440 | <.0001 |
| vs Controls | v2 | 0.3391 | 0.1056 | 0.1704 | 0.6137 | <.0001 | 1.4040 | 1.1860 | 1.8470 | <.0001 |
| | v3 | 1.7550 | 0.5185 | 0.9067 | 3.3932 | <.0001 | 5.7830 | 2.4760 | 29.7600 | <.0001 |
| Early Stage | v1 | 1.0088 | 0.2647 | 0.5395 | 1.6744 | <.0001 | 2.7420 | 1.7150 | 5.3350 | <.0001 |
| Cases | v2 | 0.3382 | 0.1059 | 0.1685 | 0.6130 | <.0001 | 1.4020 | 1.1840 | 1.8460 | <.0001 |
| vs Controls | v3 | 1.7322 | 0.5222 | 0.8760 | 3.3758 | <.0001 | 5.6530 | 2.4010 | 29.2470 | <.0001 |
| Late Stage | v1 | 1.2275 | 0.7153 | 0.2716 | 3.7556 | 0.0020 | 3.4130 | 1.3120 | 42.7580 | 0.0020 |
| Cases | v2 | The maximum likelihood estimate does not exist. | | | | | | | | |
| vs Controls | v3 | The maximum likelihood estimate does not exist. | | | | | | | | |
| Adeno | v1 | 1.1482 | 0.3927 | 0.5155 | 2.2734 | <.0001 | 3.1520 | 1.6740 | 9.7120 | <.0001 |
| Cases vs | v2 | 0.2859 | 0.1007 | 0.1282 | 0.5547 | <.0001 | 1.3310 | 1.1370 | 1.7410 | <.0001 |
| Controls | v3 | The maximum likelihood estimate does not exist. | | | | | | | | |
| Squamous | v1 | 0.8048 | 0.2638 | 0.3344 | 1.4731 | <.0001 | 2.2360 | 1.3970 | 4.3630 | <.0001 |
| Cases | v2 | 0.5531 | 0.3826 | 0.1435 | 2.1893 | <.0001 | 1.7390 | 1.1540 | 8.9290 | <.0001 |
| vs Controls | v3 | 1.4293 | 0.5076 | 0.6000 | 3.0679 | <.0001 | 4.1760 | 1.8220 | 21.4970 | <.0001 |

Samples containing “F”

| All Cases | v1 | 1.0059 | 0.2740 | 0.5276 | 1.7048 | <.0001 | 2.7340 | 1.6950 | 5.5000 | <.0001 |
|---|---|---|---|---|---|---|---|---|---|---|
| vs Controls | v2 | 0.2345 | 0.0655 | 0.1236 | 0.3873 | <.0001 | 1.2640 | 1.1320 | 1.4730 | <.0001 |
| | v3 | 1.5524 | 0.4523 | 0.7837 | 2.8341 | <.0001 | 4.7230 | 2.1900 | 17.0150 | <.0001 |
| Early Stage | v1 | 9.76E−01 | 0.2744 | 0.4963 | 1.6751 | <.0001 | 2.654 | 1.643 | 5.339 | <.0001 |
| Cases | v2 | 0.2275 | 0.0652 | 0.1171 | 0.3793 | <.0001 | 1.255 | 1.124 | 1.461 | <.0001 |
| vs Controls | v3 | 1.5232 | 0.4551 | 0.7487 | 2.8119 | <.0001 | 4.587 | 2.114 | 16.642 | <.0001 |
| Late Stage | v1 | 1.2969 | 0.7364 | 0.2878 | 3.7751 | 0.0020 | 3.6580 | 1.3330 | 43.6000 | 0.0020 |
| Cases | v2 | 0.2157 | 0.1057 | 0.0525 | 0.5761 | 0.0030 | 1.2410 | 1.0540 | 1.7790 | 0.0030 |
| vs Control | v3 | The maximum likelihood estimate does not exist. | | | | | | | | |
| Adeno | v1 | 1.2835 | 0.5302 | 0.4854 | 2.9856 | <.0001 | 3.6090 | 1.6250 | 19.7990 | <.0001 |
| Cases vs | v2 | 0.1799 | 0.0603 | 0.0801 | 0.3210 | <.0001 | 1.1970 | 1.0830 | 1.3780 | <.0001 |
| Controls | v3 | The maximum likelihood estimate does not exist. | | | | | | | | |
| Squamous | v1 | 0.7730 | 0.2611 | 0.3129 | 1.4375 | <.0001 | 2.1660 | 1.3670 | 4.2100 | <.0001 |
| Cases | v2 | 0.3100 | 0.1339 | 0.1147 | 0.6713 | <.0001 | 1.3630 | 1.1220 | 1.9570 | <.0001 |
| vs Controls | v3 | 1.2000 | 0.4220 | 0.4906 | 2.4114 | <.0001 | 3.3200 | 1.6330 | 11.1500 | <.0001 |

Multivariate exact logistic regression was attempted using the following model:

logit(status_{cass:control})=β₀+β₁v₁+β₂v₂+β₃v₃.

However, this multivariate model combining the three variables together was problematic because the program drops an individual even when one of the V1 to V3 value is missing. With small samples, it leads to unstable estimates. Importantly, in the univariate analyses, V1 to V3 were all significant in all different subsets (e.g., early state, late stage, adeno and squamous).

### Example 2—Results

Data. All of the 74 samples are used including 15 pairs labeled “C” and “F” and 11 younger controls. The clustering was done on the three variables v1, v2 and v3.

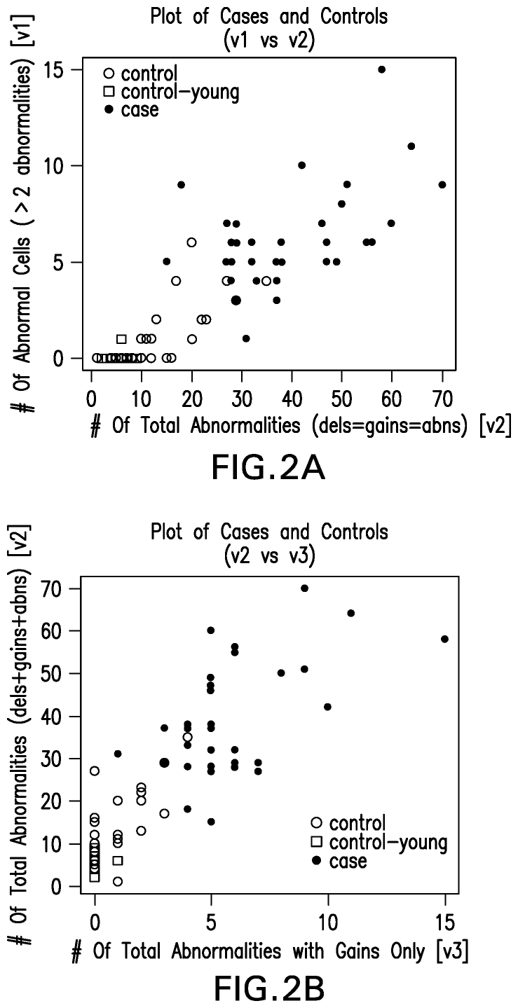

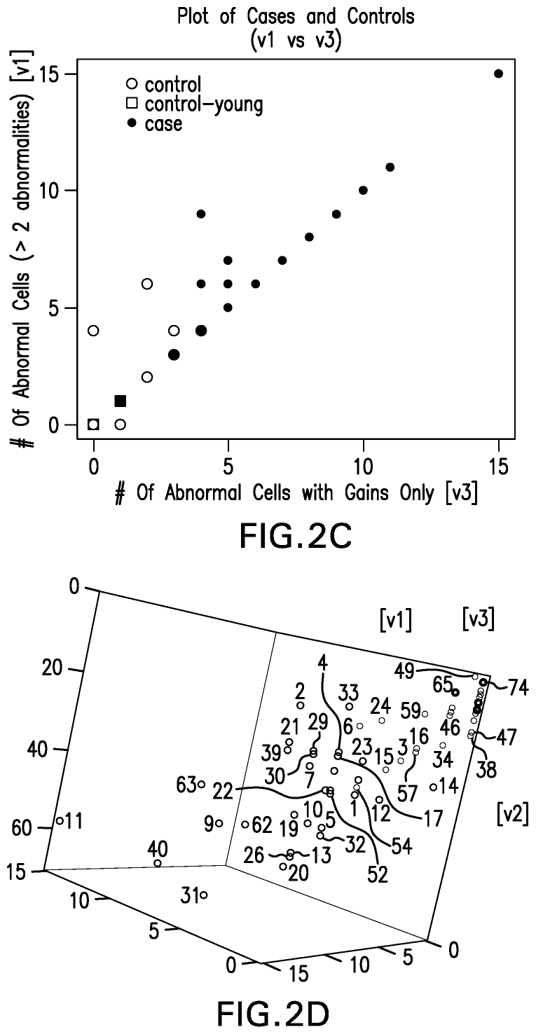

Exploration. From the scatter plots (FIGS. 2A-D), the 11 younger controls (€) are distinctively separated from all cases (●), narrowly spread in the corner. But there are a few old controls (∘) intertwining with cases in each of the three variables. This could pose a challenge in clustering analysis.

Clustering Analyses. Using SAS PROC CLUSTER, 8 of 11 clustering methods were used to the data. And the results below were extracted from the best one using Average Linkage Method, which was supported by the results from other Centroid method and Gower's Median method.

Three major statistics were examined for estimation of number of clusters, although the data set itself is defined in the groups of case (using value 3) and controls (younger group using value 2, and older group using value 1). The three statistics are the cubic clustering criterion (CCC), pseudo F (PSF), and t² (PST2).
Peak(s) with CCC value greater than 2 or 3 implies valid clusters. Great negative CCC value points to outliers.
Large number in PSF implies good numbers of clusters.
PST2 could be here as supporting result in integration with the above two to judging number of clusters. In the PsTsq chart (charting this PST2 values), the number after a pump (to the right) may indicate the number of good clusters.

TABLE 6
CLUSTER by aff (affection status for
case/control)

| | aff | | | |
|---|---|---|---|---|
| CLUSTER | 1 | 2 | 3 | Total |
| 1 | 24 | 10 | 2 | 36 |
| 2 | 3 | 0 | 20 | 23 |
| 3 | 0 | 0 | 13 | 13 |
| Total | 27 | 10 | 35 | 72 |

Frequency Missing = 2

The peak estimation of number of clusters is 3 and total 11 clusters were suggested in the data. Examination of results based on 3-cluster showed: aff=1 (younger control), 2 (old control) and 3 (case). Three controls were classified into clusters 2 where majority is cases (20 of aff=3) and 2 of the cases were grouped into cluster 1 where most controls sit. These are ambiguous samples in the clustering.

TABLE 7
Ambiguous Samples in the Clusters

| Obs | no2 | v1 | v2 | v3 | CLUSTER | aft | ID |
|---|---|---|---|---|---|---|---|
| 69 | 1 | 4 | 27 | 0 | 2 | 1 | VS13-102 |
| 28 | 3 | 3 | 29 | 3 | 2 | 1 | VS13-111C |
| 60 | 21 | 4 | 35 | 4 | 2 | 1 | VS13-142F |
| 67 | 41 | 9 | 18 | 4 | 1 | 3 | VS13-101 |
| 64 | 66 | 5 | 15 | 5 | 1 | 3 | VS13-126F |

In the three major clusters, the majority of cases were grouped into Cluster 1, and the majority of cases in Clusters 2 and 3. Table 8 and Table 9 list the successful classification of controls and cases respectively. The controls that have v2 no greater than 23 and v3 no greater than 2 were successfully identified. For cases, v2 is no lower than 27.

TABLE 8
Successfully Classified Controls

| v1 | v2 | v3 | CLUSTER | aff | ID | v1 | v2 | v3 | CLUSTER | aff | ID |
|---|---|---|---|---|---|---|---|---|---|---|---|
| 6 | 20 | 2 | 1 | 1 | VS13-105 | 0 | 1 | 1 | 1 | 1 | VS13-138 |
| 2 | 22 | 2 | 1 | 1 | VS13-111F | 0 | 2 | 0 | 1 | 2 | VS14-H-13 |
| 4 | 17 | 3 | 1 | 1 | VS13-119 | 0 | 4 | 0 | 1 | 1 | VS13-134C |
| 0 | 10 | 0 | 1 | 1 | VS13-120 | 0 | 4 | 0 | 1 | 2 | VS14-H-5 |
| 1 | 20 | 1 | 1 | 1 | VS13-127C | 0 | 4 | 0 | 1 | 2 | VS14-H-12 |
| 1 | 11 | 1 | 1 | 1 | VS13-128 | 0 | 5 | 0 | 1 | 1 | VS13-143 |
| 1 | 10 | 1 | 1 | 1 | VS13-129 | 0 | 6 | 0 | 1 | 1 | VS13-145C |
| 0 | 16 | 0 | 1 | 1 | VS13-130 | 0 | 6 | 0 | 1 | 2 | VS14-H-9 |
| 0 | 12 | 0 | 1 | 1 | VS13-132 | 1 | 6 | 1 | 1 | 2 | VS14-H-2 |
| 0 | 4 | 0 | 1 | 1 | VS13-134C | 0 | 7 | 0 | 1 | 2 | VS14-H-4 |
| 0 | 8 | 0 | 1 | 1 | VS13-134F | 0 | 7 | 0 | 1 | 2 | VS14-H-6 |
| 1 | 12 | 1 | 1 | 1 | VS13-136C | 0 | 7 | 0 | 1 | 2 | VS14-H-10 |
| 0 | 15 | 0 | 1 | 1 | VS13-136F | 0 | 8 | 0 | 1 | 1 | VS13-134F |
| 0 | 1 | 1 | 1 | 1 | VS13-138 | 0 | 8 | 0 | 1 | 1 | VS13-142C |
| 0 | 12 | 0 | 1 | 1 | VS13-139 | 0 | 8 | 0 | 1 | 2 | VS14-H-1 |
| 0 | 10 | 0 | 1 | 1 | VS13-140 | 0 | 9 | 0 | 1 | 2 | VS14-H-3 |
| 0 | 8 | 0 | 1 | 1 | VS13-142C | 0 | 10 | 0 | 1 | 1 | VS13-120 |
| 0 | 5 | 0 | 1 | 1 | VS13-143 | 0 | 10 | 0 | 1 | 1 | VS13-140 |
| 0 | 12 | 0 | 1 | 1 | VS13-144C | 1 | 10 | 1 | 1 | 1 | VS13-129 |
| 2 | 23 | 2 | 1 | 1 | VS13-144F | 1 | 10 | 1 | 1 | 1 | VS13-146 |
| 0 | 6 | 0 | 1 | 1 | VS13-145C | 1 | 10 | 1 | 1 | 1 | VS13-147 |
| 2 | 13 | 2 | 1 | 1 | VS13-145F | 1 | 11 | 1 | 1 | 1 | VS13-128 |
| 1 | 10 | 1 | 1 | 1 | VS13-146 | 0 | 12 | 0 | 1 | 1 | VS13-132 |
| 1 | 10 | 1 | 1 | 1 | VS13-147 | 0 | 12 | 0 | 1 | 1 | VS13-139 |
| 0 | 8 | 0 | 1 | 2 | VS14-H-1 | 0 | 12 | 0 | 1 | 1 | VS13-144C |
| 0 | 7 | 0 | 1 | 2 | VS14-H-10 | 1 | 12 | 1 | 1 | 1 | VS13-136C |
| 0 | 4 | 0 | 1 | 2 | VS14-H-12 | 2 | 13 | 2 | 1 | 1 | VS13-145F |
| 0 | 2 | 0 | 1 | 2 | VS14-H-13 | 0 | 15 | 0 | 1 | 1 | VS13-136F |
| 1 | 6 | 1 | 1 | 2 | VS14-H-2 | 0 | 16 | 0 | 1 | 1 | VS13-130 |
| 0 | 9 | 0 | 1 | 2 | VS14-H-3 | 4 | 17 | 3 | 1 | 1 | VS13-119 |
| 0 | 7 | 0 | 1 | 2 | VS14-H-4 | 1 | 20 | 1 | 1 | 1 | VS13-127C |
| 0 | 4 | 0 | 1 | 2 | VS14-H-5 | 6 | 20 | 2 | 1 | 1 | VS13-105 |
| 0 | 7 | 0 | 1 | 2 | VS14-H-6 | 2 | 22 | 2 | 1 | 1 | VS13-111F |
| 0 | 6 | 0 | 1 | 2 | VS14-H-9 | 2 | 23 | 2 | 1 | 1 | VS13-144F |

Note:
“aff” stands for affection status for case and control. Value 2 represents younger controls. The left-sided table is sorted by ID, and the right-sided table is sorted by v2, v1 and v3.

TABLE 9
Successfully Classified Cases
The left-sided table is sorted by ID, and the right-sided table is sorted by v2, v1 and v3

| v1 | v2 | v3 | CLUSTER | aff | ID | v1 | v2 | v3 | CLUSTER | aff | ID |
|---|---|---|---|---|---|---|---|---|---|---|---|
| 4 | 37 | 4 | 2 | 3 | VS13-100 | 5 | 27 | 5 | 2 | 3 | VS13-103 |
| 5 | 27 | 5 | 2 | 3 | VS13-103 | 7 | 27 | 7 | 2 | 3 | VS13-116F |
| 5 | 47 | 5 | 3 | 3 | VS13-104 | 7 | 27 | 7 | 2 | 3 | VS13-122 |
| 6 | 32 | 6 | 2 | 3 | VS13-106 | 4 | 28 | 4 | 2 | 3 | VS13-107C |
| 4 | 28 | 4 | 2 | 3 | VS13-107C | 4 | 28 | 4 | 2 | 3 | VS13-118 |
| 9 | 51 | 9 | 3 | 3 | VS13-107F | 5 | 28 | 5 | 2 | 3 | VS13-112 |
| 6 | 47 | 5 | 3 | 3 | VS13-108C | 6 | 28 | 6 | 2 | 3 | VS13-124 |
| 15 | 58 | 15 | 3 | 3 | VS13-108F | 3 | 29 | 3 | 2 | 3 | VS13-137 |
| 3 | 37 | 3 | 2 | 3 | VS13-109C | 6 | 29 | 6 | 2 | 3 | VS13-125C |
| 6 | 55 | 6 | 3 | 3 | VS13-109F | 7 | 29 | 7 | 2 | 3 | VS13-131C |
| 1 | 31 | 1 | 2 | 3 | VS13-110 | 1 | 31 | 1 | 2 | 3 | VS13-110 |
| 5 | 28 | 5 | 2 | 3 | VS13-112 | 5 | 32 | 5 | 2 | 3 | VS13-135 |
| 5 | 37 | 5 | 2 | 3 | VS13-113 | 6 | 32 | 6 | 2 | 3 | VS13-106 |
| 7 | 46 | 5 | 3 | 3 | VS13-115 | 4 | 33 | 4 | 2 | 3 | VS13-123 |
| 7 | 60 | 5 | 3 | 3 | VS13-116C | 4 | 33 | 4 | 2 | 3 | VS13-133 |
| 7 | 27 | 7 | 2 | 3 | VS13-116F | 3 | 37 | 3 | 2 | 3 | VS13-109C |
| 6 | 38 | 4 | 2 | 3 | VS13-117 | 4 | 37 | 4 | 2 | 3 | VS13-100 |
| 4 | 28 | 4 | 2 | 3 | VS13-118 | 5 | 37 | 5 | 2 | 3 | VS13-113 |
| 6 | 56 | 6 | 3 | 3 | VS13-121 | 5 | 38 | 5 | 2 | 3 | VS13-141 |
| 7 | 27 | 7 | 2 | 3 | VS13-122 | 6 | 38 | 4 | 2 | 3 | VS13-117 |
| 4 | 33 | 4 | 2 | 3 | VS13-123 | 10 | 42 | 10 | 3 | 3 | VS13-148F |
| 6 | 28 | 6 | 2 | 3 | VS13-124 | 7 | 46 | 5 | 3 | 3 | VS13-115 |
| 6 | 29 | 6 | 2 | 3 | VS13-125C | 5 | 47 | 5 | 3 | 3 | VS13-104 |
| 9 | 70 | 9 | 3 | 3 | VS13-125F | 6 | 47 | 5 | 3 | 3 | VS13-108C |
| 5 | 49 | 5 | 3 | 3 | VS13-126C | 5 | 49 | 5 | 3 | 3 | VS13-126C |
| 7 | 29 | 7 | 2 | 3 | VS13-131C | 8 | 50 | 8 | 3 | 3 | VS13-148C |
| 11 | 64 | 11 | 3 | 3 | VS13-131F | 9 | 51 | 9 | 3 | 3 | VS13-107F |
| 4 | 33 | 4 | 2 | 3 | VS13-133 | 6 | 55 | 6 | 3 | 3 | VS13-109F |
| 5 | 32 | 5 | 2 | 3 | VS13-135 | 6 | 56 | 6 | 3 | 3 | VS13-121 |
| 3 | 29 | 3 | 2 | 3 | VS13-137 | 15 | 58 | 15 | 3 | 3 | VS13-108F |
| 5 | 38 | 5 | 2 | 3 | VS13-141 | 7 | 60 | 5 | 3 | 3 | VS13-116C |
| 8 | 50 | 8 | 3 | 3 | VS13-148C | 11 | 64 | 11 | 3 | 3 | VS13-131F |
| 10 | 42 | 10 | 3 | 3 | VS13-148F | 9 | 70 | 9 | 3 | 3 | VS13-125F |

TABLE 10
Dropped Samples due to missing value

| Obs | no2 | v1 | v2 | v3 | CLUSTER | aff | ID |
|---|---|---|---|---|---|---|---|
| 1 | 8 | 0 | 7 | | | 1 | VS13-127F |
| 2 | 35 | 0 | 3 | | | 2 | VS14-H-7 |

Euclidean Distance to Origin. Another simple measurement is attempted to calculate the squared Euclidean distance to origin of each sample, “EuclideanD”, using the formula: EuclideanD=(v1)²+(v2)²+(v3)², e.g., for example, in the case of sample ID=VS13-105, which has v1=6, v2=20 and v3=2, its EuclideanD=6²+20²+2²=36+400+4=440.

The EuclideanDs are listed in Tables 11 (successfully classified controls), 12 (successfully classified cases) and 13 (misclassified cases/controls). For controls with EuclideanD <=537 or cases with EuclideanD >=779 the classification appears to be successful.

In Table 13, there is one control, sample ID=VS13-101, being grouped in Cluster 2. This sample has EuclideanD=859, exactly the same value as the case sample ID=VS13-137 (in Cluster 2 as well). The two samples share exactly the same values v1(=3), v2(=29) and v3(=3). The Average Linkage clustering method grouped them both in Cluster 2, but one was identified as a control and thus considered as a misclassification.

TABLE 11
EuclideanD in Successfully Classified Controls

| Obs | no2 | v1 | v2 | v3 | CLUSTER | aff | ID | EucolideanD |
|---|---|---|---|---|---|---|---|---|
| 49 | 17 | 0 | 1 | 1 | 1 | 1 | VS13-138 | 2 |
| 50 | 39 | 0 | 2 | 0 | 1 | 2 | VS14-H-13 | 4 |
| 14 | 13 | 0 | 4 | 0 | 1 | 1 | VS13-134C | 16 |
| 15 | 33 | 0 | 4 | 0 | 1 | 2 | VS14-H-5 | 16 |
| 21 | 38 | 0 | 4 | 0 | 1 | 2 | VS14-H-12 | 16 |
| 33 | 22 | 0 | 5 | 0 | 1 | 1 | VS13-143 | 25 |
| 18 | 25 | 0 | 6 | 0 | 1 | 1 | VS13-145C | 36 |
| 19 | 36 | 0 | 6 | 0 | 1 | 2 | VS14-H-9 | 36 |
| 48 | 30 | 1 | 6 | 1 | 1 | 2 | VS14-H-2 | 38 |
| 16 | 32 | 0 | 7 | 0 | 1 | 2 | VS14-H-4 | 49 |
| 17 | 34 | 0 | 7 | 0 | 1 | 2 | VS14-H-6 | 49 |
| 20 | 37 | 0 | 7 | 0 | 1 | 2 | VS14-H-10 | 49 |
| 7 | 14 | 0 | 8 | 0 | 1 | 1 | VS13-134F | 64 |
| 8 | 20 | 0 | 8 | 0 | 1 | 1 | VS13-142C | 64 |
| 13 | 29 | 0 | 8 | 0 | 1 | 2 | VS14-H-1 | 64 |
| 36 | 31 | 0 | 9 | 0 | 1 | 2 | V514-H-3 | 81 |
| 5 | 6 | 0 | 10 | 0 | 1 | 1 | V513-120 | 100 |
| 6 | 19 | 0 | 10 | 0 | 1 | 1 | V513-140 | 100 |
| 10 | 10 | 1 | 10 | 1 | 1 | 1 | V513-129 | 102 |
| 11 | 27 | 1 | 10 | 1 | 1 | 1 | V513-146 | 102 |
| 12 | 28 | 1 | 10 | 1 | 1 | 1 | V513-147 | 102 |
| 30 | 9 | 1 | 11 | 1 | 1 | 1 | V513-128 | 123 |
| 3 | 12 | 0 | 12 | 0 | 1 | 1 | V513-132 | 144 |
| 4 | 18 | 0 | 12 | 0 | 1 | 1 | V513-139 | 144 |
| 9 | 23 | 0 | 12 | 0 | 1 | 1 | V513-144C | 144 |
| 47 | 15 | 1 | 12 | 1 | 1 | 1 | V513-136C | 146 |
| 65 | 26 | 2 | 13 | 2 | 1 | 1 | V513-145F | 177 |
| 32 | 16 | 0 | 15 | 0 | 1 | 1 | V513-136F | 225 |
| 31 | 11 | 0 | 16 | 0 | 1 | 1 | V513-130 | 256 |
| 63 | 5 | 4 | 17 | 3 | 1 | 1 | V513-119 | 314 |
| 62 | 7 | 1 | 20 | 1 | 1 | 1 | V513-127C | 402 |
| 66 | 2 | 6 | 20 | 2 | 1 | 1 | V513-105 | 440 |
| 34 | 4 | 2 | 22 | 2 | 1 | 1 | V513-111F | 492 |
| 35 | 24 | 2 | 23 | 2 | 1 | 1 | V513-144F | 537 |

TABLE 12
EuclideanD in Successfully Classified Cases

| Obs | no2 | v1 | v2 | v3 | CLUSTER | aff | ID | EucolideanD |
|---|---|---|---|---|---|---|---|---|
| 39 | 42 | 5 | 27 | 5 | 2 | 3 | VS13-103 | 779 |
| 22 | 45 | 4 | 28 | 4 | 2 | 3 | VS13-107C | 816 |
| 23 | 58 | 4 | 28 | 4 | 2 | 3 | VS13-118 | 816 |
| 24 | 56 | 7 | 27 | 7 | 2 | 3 | VS13-116F | 827 |
| 25 | 60 | 7 | 27 | 7 | 2 | 3 | VS13-122 | 827 |
| 40 | 52 | 5 | 28 | 5 | 2 | 3 | VS13-112 | 834 |
| 43 | 62 | 6 | 28 | 6 | 2 | 3 | VS13-124 | 856 |
| 29 | 71 | 3 | 29 | 3 | 2 | 3 | VS13-137 | 859 |
| 44 | 63 | 6 | 29 | 6 | 2 | 3 | VS13-125C | 913 |
| 56 | 67 | 7 | 29 | 7 | 2 | 3 | VS13-131C | 939 |
| 70 | 51 | 1 | 31 | 1 | 2 | 3 | VS13-110 | 963 |
| 54 | 70 | 5 | 32 | 5 | 2 | 3 | VS13-135 | 1074 |
| 53 | 44 | 6 | 32 | 6 | 2 | 3 | VS13-106 | 1096 |
| 26 | 61 | 4 | 33 | 4 | 2 | 3 | VS13-123 | 1121 |
| 27 | 69 | 4 | 33 | 4 | 2 | 3 | VS13-133 | 1121 |
| 52 | 49 | 3 | 37 | 3 | 2 | 3 | VS13-109C | 1387 |
| 51 | 40 | 4 | 37 | 4 | 2 | 3 | VS13-100 | 1401 |
| 45 | 53 | 5 | 37 | 5 | 2 | 3 | VS13-113 | 1419 |
| 46 | 72 | 5 | 38 | 5 | 2 | 3 | VS13-141 | 1494 |
| 55 | 57 | 6 | 38 | 4 | 2 | 3 | VS13-117 | 1496 |
| 73 | 74 | 10 | 42 | 10 | 3 | 3 | VS13-148F | 1964 |
| 59 | 54 | 7 | 46 | 5 | 3 | 3 | VS13-115 | 2190 |
| 37 | 43 | 5 | 47 | 5 | 3 | 3 | VS13-104 | 2259 |
| 38 | 47 | 6 | 47 | 5 | 3 | 3 | VS13-108C | 2270 |
| 61 | 65 | 5 | 49 | 5 | 3 | 3 | VS13-126C | 2451 |
| 58 | 73 | 8 | 50 | 8 | 3 | 3 | VS13-148C | 2628 |
| 57 | 46 | 9 | 51 | 9 | 3 | 3 | VS13-107F | 2763 |
| 41 | 50 | 6 | 55 | 6 | 3 | 3 | VS13-109F | 3097 |
| 42 | 59 | 6 | 56 | 6 | 3 | 3 | VS13-121 | 3208 |
| 68 | 55 | 7 | 60 | 5 | 3 | 3 | VS13-116C | 3674 |
| 74 | 48 | 15 | 58 | 15 | 3 | 3 | VS13-108F | 3814 |
| 72 | 68 | 11 | 64 | 11 | 3 | 3 | VS13-131F | 4338 |
| 71 | 64 | 9 | 70 | 9 | 3 | 3 | VS13-125F | 5062 |

TABLE 13
Misclassified Cases and Controls

| Obs | no2 | v1 | v2 | v3 | CLUSTER | aff | ID | EucolideanD |
|---|---|---|---|---|---|---|---|---|
| 64 | 66 | 5 | 15 | 5 | 1 | 3 | VS13-102 | 275 |
| 67 | 41 | 9 | 18 | 4 | 1 | 3 | VS13-111C | 421 |
| 69 | 1 | 4 | 27 | 0 | 2 | 1 | VS13-142F | 745 |
| 28 | 3 | 3 | 29 | 3 | 2 | 1 | VS13-101 | 859 |
| 60 | 21 | 4 | 35 | 4 | 2 | 1 | VS13-126F | 1257 |

Using this system, the inventor was able to enrich the numbers of abnormal cells in a subgroup of patients (9) and controls (5) whose blood specimens were reanalyzed on the Bioview system using this new threshold to enrich the malignant cell population). Of the 9 cancer cases so measured, (most with early lung cancer), 5/9 showed an increase in abnormal cells (defined as a polysomy or gain of 2 chormosomal loci within the same cell). In one case of early adenocarcinoma initially scored as negative for malignancy (based on a threshold for positivity of 4 or > abnormal cells), the reclassification with the new software raised the number of abnormal cells from 3 to 5 (Case V13-109; see below) thus enabling us to classify this case as positive and improve sensitivity and specificity for the test.

Whole screen shots were taken showing circulating tumor cells (ctc) enrichment before and after soft-ware optimization. Tumor cell or circulating tumor cell (ctc) was defined as any cell with 2 or > chromosomal gains or polysomies per cell. This approach results in a minimum of 10 fish signals per cell, and a positive case is defined as a blood sample with 4 or > ctc's. Three representative cases are discussed below:
VS13-109. A 45 year-old female, a former smoker, with Stage IB Squamous Cell Lung Cancer showed 3 abnormal cells prior to optimization of Software on Duet (Bioview Ltd.) using a four-color FISH panel. After optimization, abnormal cells from the same case were observed using a four-color FISH panel (increase of 66% in abnormal cell detection).
VS13-121. An 82 year-old male, a current smoker who was diagnosed with Stage IIB Adenocarcinoma of Lung showed 6 FISH-abnormal Cells with a four-color FISH panel prior to optimization of the Software on Duet (Bioview Ltd.). After optimization, 10 FISH-abnormal cells were observed with a four-color FISH panel (increase of 66% in abnormal cell detection).
VS13-124. A 40 year-old male, a former smoker diagnosed with Stage IIA Squamous Cell Lung Cancer, showed 6 abnormal Cells prior to optimization of the Software on Duet, Bioview Ltd. using a four-color FISH panel. Post-optimization of software, abnormal cell yield increased to 8 abnormal cells (33% increase) using a four-color FISh panel.
   Table 14 summarizes the data for these 5 patients with lung cancer whose abnormal (malignant) circulating tumor cell numbers increased with the new software modification (old numbers are in parentheses).

TABLE 14

| | #ABNORMAL CELLS | |
|---|---|---|
| CASE NUMBER | New | Old |
| Case V13-109 | 5 | 3 |
| Case V13-124 | 8 | 6 |
| Case VS 13-121 | 10 | 6 |
| Case V13-117 | 9 | 4 |
| Case V13-135 | 7 | 5 |

In control cases, the numbers of abnormal cells were not increased by no more than 1 cell, however remained far below the threshold of 4, which is the threshold for malignancy.

Thyroid cancer. Enumeration of circulating tumor cells (CTCs) in papillary thyroid cancer (PTC) patients has been unsuccessful, when based on EpCAM assays which failed to capture CTCs undergoing epithelial to mesenchymal transition. To overcome this, the inventor designed an antigen-independent FISH based assay using DNA probes that had previously been used to detect lung cancer CTCs. The inventor hypothesized that these same probes discussed above might detect CTCs in PTC, as both lung and thyroid are derived from foregut endoderm and associated with NKX2-1/TTF1, a lineage-survival oncogene, which controls expression of genes such as surfactant proteins (SFPT), that are associated with differentiation in lung and thyroid progenitor cells. In addition it has been demonstrated by RNAseq that the majority PTCs overexpress surfactant protein A, B and C RNA.

To evaluate the presence of CTCs in peripheral blood of patients with metastatic PTC (PTCM), the inventor tested patients with PTCM, and controls, in an attempt to establish a new prognostic/surrogate marker of disease progression and response to therapy. She also sought to see if the lung cancer probe set could accurately detect CTCs of PTC. Twelve PTCM patients (aged 62±15.7 years) and 8 control patients (aged 46.9±12.1 years) with history of PTC, successful thyroidectomy and no evidence of disease (NED) for more than 5 years were recruited. Thirty healthy subjects (aged 62.7±7.8 year) were included as a second control group. Peripheral blood mononuclear cells (PBMCs) were isolated and hybridized with a multi-color cocktail of 4 DNA probes, 2 locus specific probes, at 10q22 (SFTPA1, 2) and 3p22 and 2 internal centromeric probes, CEP10 and CEP 3. A scanning system scored fluorescent signals on a per cell basis on 500 cells per sample. Signal patterns were analyzed by 2 readers into classes: abnormal cells (AC) or CTCs (gains of 2 or > probes); deletions or gains (loss or gain of a single probe), and normal cells.

Patients with PTCM had higher % ACs (0.9±0.3, P<0.001 and 99.9% power) than patients with NED (0.18±0.18) or healthy controls (0.18±0.23). The cutoff of 0.6% AC (3/500 cells) differentiated between PTCM and controls from both groups. Compared to healthy controls, patients with PTCM had higher percentages of del of CEP 3 (0.80±0.85 vs 0.19±0.53, P=0.006), CEP10 (1.43±1.37 vs 0.45±0.67, P=0.002), gain of CEP10 (0.45±0.34 vs 0.23±0.28, P=0.03) and gain of 10q22.3 (0.77±0.60 vs 0.13±0.17, P<0.001), total deletions and gains (5.68±1.91 vs 2.47±1.27, P<0.001) and decreased % normal cells (94.33±1.88 vs 97.52±1.24 (P<0.00001).

PTCM demonstrated CTCs characterized by aneuploidy, with higher levels of CTCs compared to controls. The probes designed for lung cancer were successful in detecting genetic aberrations in PTC patients' CTCs, likely as a result of a common lineage-specific transcription factor controlling expression of genes, in lung and thyroid malignant progenitor cells. Studies with larger cohorts are needed to confirm the significance of CTCs in the prognosis of PTCM.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Pat. No. 4,415,723
U.S. Pat. No. 4,458,066
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,610,287
U.S. Pat. No. 5,739,169
U.S. Pat. No. 5,795,715
U.S. Pat. No. 5,801,005
U.S. Pat. No. 5,824,311
U.S. Pat. No. 5,830,880
U.S. Pat. No. 5,846,945
U.S. Pat. No. 5,889,136
U.S. Pat. No. 6,506,559
U.S. Pat. No. 6,573,099
U.S. Pat. No. 6,797,471
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0064842
U.S. Patent Publn. 2004/0265839
U.S. Patent Publn. 2007/0218480
U.S. Patent Publn. 2009/0137412
Alberola et al., Proc. Ann. Mt. Am. Soc. Clin. Oncol., 14: A1094, 1995.
Allard et al., Clin. Cancer Res., 10:6897-6904, 2004.
Arap et al., Cancer Res., 55(6):1351-1354, 1995.
Auerbach et al., N. Engl J. Med., 265: 253-267, 1961.
Austin-Ward and Villaseca, Revista Medica de Chile, 126(7):838-845, 1998.
Ayabe et al., Lung Cancer, 11(3-4): 201-208, 1994.
Bakhshi et al., Cell, 41(3):899-906, 1985.
Barkan et al., Cancer, 105:35-43, 2004.
Barkan et al., Cancer, 105:35-43, 2005.
Bonassi et al., Cancer Res., 60:1619-1625, 2000.
Bonassi et al., Genome Res., 104(1-4):376-382, 2004.
Bosher and Labouesse, Nat. Cell. Biol., 2(2):E31-E36, 2000.
Brummelkamp et al., Cancer Cell, 2:243-247, 2002.
Bubendorf et al., Am. J. Clin. Pathol., 123516-123523, 2005.
Bukowski et al., Clinical Cancer Res., 4(10):2337-2347, 1998.
Caldas et al., Cancer Res., 54:3568-3573, 1994.
Carcy et al., JNCI, 65: 1225-1230, 1980.
Carriaga et al., Cancer, 75: 406-421, 1995.
Cheng et al., Cancer Res., 54(21):5547-5551, 1994.
Cheon et al., Yonsei Med J., 34(4): 365-370, 1993.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cristofanilli et al., N. Engl. J. Med., 351:781-791, 2004.
Daniely et al., Urology, 66:1354-1359, 2005.
Davidson et al., J. Immunother., 21(5):389-398, 1998.
Ekins and Chu, Trends in Biotechnology, 17: 217-218, 1999.
EPO Appln. 320 308
EPO Appln. 329 822
Feder et al., Cancer Genet. Cytogenet., 102: 25-31, 1998.
Feinstein et al., Am. Rev. Repir. Dis., 101: 671-684, 1970.
Fenech and Crott, Mutat. Res., 504:131-136, 2002.
Fenech, Drug Discov. Today, 7:1128-1137, 2002.
Fenech, Mutat. Res., 455:81-95, 2000.
Fenech, Toxicology, 181-182:411-416, 2002.
Fire et al., Nature, 391(6669):806-811, 1998.
Fodor et al., Biochemistry, 30(33):8102-8108, 1991.
Fontanini et al., Cancer, 70(6): 1520-7, 1992.
Freifelder, In: Physical Biochemistry Applications to Biochemistry and Molecular Biology, 2nd Ed. Wm. Freeman and Co., NY, 1982.
Frohman, In: PCR Protocols: A Guide To Methods And Applications, Academic Press, N.Y., 1990.
Gangnus et al., Clin. Cancer Res., 10:3457-3464, 2004.
GB Appln. 2 202 328
Goeze et al., J. Pathol., 196:8-16, 2002.
Gough et al., Biomed. Central, 4:9:1471-2164, 2003.
Grishok et al., Science, 287:2494-2497, 2000.
Haagsman and Diemel, Comp. Biochem. Physiol. Mol. Integr. Physiol., 129(1):91-108, 2001.
Hacia et al., Nature Genet., 14:441-449, 1996.
Hagmar et al., Cancer Res., 58:4117-4121, 1998.
Hanibuchi et al., Int. J. Cancer, 78(4):480-485, 1998.
Hellstrand et al., Acta Oncologica, 37(4):347-353, 1998.
Hirano et al., American J. Path., 144(2): 296-302, 1994.
Hirsh, Manksgaard, 1-78, 1983.
Holmstrom et al., Anal. Biochem. 209:278-283, 1993.
Huang et al., Proc. Nat'l Acad. Sci. USA 98: 15044-15049, 2001.
Hui and Hashimoto, Infection Immun., 66(11):5329-5336, 1998.
Hussussian et al., Nat. Genet., 8(1):15-21, 1994.
Ichinose et al., Cancer Res., 51(19):5392-5397, 1991.
Ihde, Curr. Prob. Cancer, 15: 65, 1991.
Ingenito et al., J. Appl. Physiol., 86(5):1702-1714, 1999.
Innis, et al., In: PCR Protocols. A Guide to Methods and Application, Academic Press, Inc. San Diego, 1990.
Jiang and Katz, Diagn. Mol. Pathol., 11:47-57, 2002.
Jiang et al., Clin. Cancer Res., 115417-115424, 2005.
Jiang et al., Neoplasia, 6:623-635, 2004.
Jones, Nature, 199:280-282, 1963.
Ju et al., Gene Ther., 7(19):1672-1679, 2000.
Kamb et al., Nat. Genet., 8(1):23-26, 1994.
Kamb et al., Science, 2674:436-440, 1994.
Katz et al., Mod. Pathol., 21:950-960, 2008.
Kerr et al., Br. J. Cancer, 26(4):239-257, 1972.
Ketting et al., Cell, 99(2):133-141, 1999.
Kim et al., Cell, 87:343-355, 1996.
Korfhagen et al., Proc. Natl. Acad. Sci. USA, 93(18):9594-9599, 1996.
Kwoh et al., Proc. Natl. Acad. Sci. USA, 86:1173, 1989.
Lee et al., Biochim. Biophys. Acta, 1582:175-177, 2002.
Leversha et al., Clin. Cancer Res., 15(6):2091-7, 2009.
Li et al., Lab. Invest., 86(6):619-627, 2006.
Licciardello et al., Int. J. Radiat. Oncol. Biol. Phys., 17(3):467-476, 1989.
Liewald et al., Chirurg., 63(3): 205-10, 1992.
Lin and Avery, Nature, 402:128-129, 1999.
Liou et al., Cancer Res., 59:1481-1484, 1999.
Macchiarini et al., Proc Annu Mt. Am. Soc. Clin. Oncol., 11: A995, 1992.
Mitchell et al., Ann. NY Acad. Sci., 690:153-166, 1993.
Mitchell et al., J. Clin. Oncol., 8(5):856-869, 1990.
Mitsudomi et al., Clin. Cancer Res., 2(7): 1185-9, 1996.
Miyamoto et al., Cancer Res., 51(23pt1) 6346-50, 1991.
Montgomery et al., Proc. Natl. Acad. Sci. USA, 95:15502-15507, 1998.
Mori et al., Cancer Res., 54(13):3396-3397, 1994.
Morton et al., Arch. Surg., 127:392-399, 1992.
Mrkve et al., Anticancer Research, 13(3): 571-8, 1993.
Muller and Pantel, Breast Cancer Res., 6:258-261, 2004.
Naruke et al., J. Thorac. Cardiovas Surg., 96: 400, 1988.
Newton et al., Nucl. Acids Res. 21:1155-1162, 1993.
Nobori et al., Nature (London), 368:753-756, 1995.
Ohara et al., Proc. Natl. Acad. Sci. USA, 86:5673-5677, 1989.
Okamoto et al., Proc. Natl. Acad. Sci. USA, 91(23):11045-11049, 1994.
Orlow et al., Cancer Res, 54(11):2848-2851, 1994.
Orlow et al., Int. J. Oncol., 15(1):17-24, 1994.
Palaniyar et al., Biochem. Biophys. Res. Commun., 250(1):131-136, 1998.
Palaniyar et al., Comp. Biochem. Physiol. A Mol. Integr. Physiol., 129(1):109-127, 2001.
Pantel et al., Proc. Annu Mt. Am. Soc. Clin. Oncol., 12: A941, 1993.
Papadimitrakopoulou et al., Cancer and Metastasis Reviews, 15: 53-76, 1996.
Paul et al., Nature Biotechnol., 20:505-508, 2002.
PCT Appln. WO 00/44914
PCT Appln. WO 01/36646
PCT Appln. WO 01/68836
PCT Appln. WO 88/10315
PCT Appln. WO 89/06700
PCT Appln. WO 90/07641
PCT Appln. WO 99/32619
PCT Pubin. PCT/US87/00880
PCT Pubin. PCT/US89/01025
Pease et al., Proc. Natl. Acad. Sci. USA, 91:5022-5026, 1994.
Pence et al., Archives of Surgery, 128(12): 1382-1390, 1993.
Pérez-Gil et al., Biochim. Biophys. Acta., 1408(2-3):203-217, 1998.
Pierga et al., Clin. Cancer Res., 10:1392-1400, 2004.
Pietras et al., Oncogene, 17(17):2235-2249, 1998.
Pignon et al., Hum. Mutat., 3: 126-132, 1994.
Qin et al., Proc. Natl. Acad. Sci. USA, 95(24):14411-14416, 1998.
Ramaswamy et al., Nat. Genet, 33:49-54, 2003.
Rasmussen et al., Anal. Biochem, 198:138-142, 1991.
Ravindranath and Morton, Intern. Rev. Immunol., 7: 303-329, 1991.
Rice et al., J. Thoracic Cardio. Surgery, 106(2): 201-217, 1993.
Rosenberg et al., Ann. Surg. 210(4):474-548, 1989.
Rosenberg et al., N. Engl. J. Med., 319:1676, 1988.
Running et al., BioTechniques 8:276-277, 1990.
Sahin et al., Cancer, 65(3): 530-7, 1990.
Saiki et al., J. Biochem., 116(6):1208-1211, 1994.
Sambrook et al., In: Molecular cloning: a laboratory manual, 2^{nd} Ed., Cold Spring Harbor Laboratory Press, Cold Spring Harbor, N.Y., 1989.
Serrano et al., Nature, 366:704-707, 1993.
Serrano et al., Science, 267(5195):249-252, 1995.
Sharp and Zamore, Science, 287:2431-2433, 2000.
Sharp, Genes Dev., 13:139-141, 1999.
Sher et al., Clin. Cancer Res., 11:173-179, 2005.
Shiseki et al., Genes Chromosomes Cancer, 17(2): 71-7, 1996.
Shoemaker et al., Nature Genetics, 14:450-456, 1996.
Shriver et al., Mutat. Res. 406(1):9-23, 1998.
Sieuwerts et al., J. Natl. Cancer Inst., 101:61-66, 2009.
Slaughter et al., Cancer, 6:963-968, 1953.
Smerhovsky et al., Environ. Health Perspect., 109:41-45, 2001.
Solomon et al., Science, 254:1153-1160, 1991.
Stewenius et al., Proc. Natl. Acad. Sci. USA, 102(15):5541-5546, 2005.
Sui et al., Proc. Natl. Acad. Sci. USA, 99(8):5515-5520, 2002.
Swennenhuis et al., Cytometry A, 75(6):520-7, 2009.
Tabara et al., Cell, 99(2):123-132, 1999.
Thiberville et al., Cancer Research, 55: 5133-5139, 1995a.
Thiberville et al., Int. J. Cancer, 64:371, 1995b.
Travis et al., Cancer, 75: 191-202, 1995.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
Tucker and Preston, Mutat. Res., 365:147-159, 1996.
Umegaki et al., Mutagenesis, 15:261-269, 2000.
Valdivieso et al., Proc. Annu. Mt. Am. Soc. Clin. Oncol., 13: A1121, 1994.
Vallmer et al., Hum. Pathol., 16: 247-252, 1985.
Volm et al., Versicherungsmedizin, 41(1): 2-5, 1989.
Voravud, et al., Cancer Research, 53: 2874-2883, 1993.
Walker et al., Nucleic Acids Res., 20(7):1691-1696, 1992.
Wincott et al., Nucleic Acids Res., 23(14):2677-2684, 1995.
Wistuba et al., Cancer Res., 60(7): 1949-60, 2000.
Wu et al., Anal. Chem., 70:456A, 1998.
Wu et al., Cancer Res., 58(8):1605-8, 1998.
Wu et al., J, Thorac Oncol., 4:30-36, 2009.
Yamaoka et al., J. Japan Surgical Soc., 91(10):1608-1616, 1990.
Yanagisawa et al., Cancer Research, 56:5579-5582, 1996.
Yendamuri et al., J. Thorac Oncol., 3:979-984, 2008.
Yu et al., J. Am. Chem. Soc., 124(23):6576-6583, 2002.
Zou et al., Clinical Cancer Research 4: 1345-1355, 1998.

1. A method of detecting circulating tumor cells (CTCs) in a sample containing blood cells comprising:
(a) selecting CTCs from a sample containing blood cells by assessing nuclear area and/or circularity;
(b) hybridizing the selected cells with labeled nucleic acid probes for 3p22.1, 10q22.3, chromosome 10 centromeric (cep10) and chromosome 3 centromeric (cep3);
(c) evaluating the signal pattern for the selected cells by detecting fluorescence in situ hybridization from cells; and
(d) detecting CTCs based on pattern of hybridization to all four labeled nucleic acid probes to said selected cells.

2. The method of claim 1, further comprising, prior to step (b), filtering said blood sample.

3-4. (canceled)

5. The method of claim 1, wherein the blood sample is a buffy coat layer separated from the blood by a Ficoll-Hypaque gradient.

6. The method of claim 5, wherein the buffy coat layer is further purified by CD45 bead-based purification to remove white blood cells, or wherein the buffy coat layer is further purified by CD3 bead-based purification to remove white blood cells.

7. (canceled)

8. The method of claim 1, wherein the patient is known or suspected to have cancer.

9-10. (canceled)

11. The method of claim 1, wherein the staining further comprises contacting the sample with a labeled CD45 antibody, a labeled SNAIL1 antibody, and/or a labeled anti-GLUT1 antibody.

12. (canceled)

13. The method of claim 1, wherein detecting the signal comprises using an automated fluorescence scanner.

14. The method of claim 1, further comprising using and detecting one or more additional probes in steps (b)-(d).

15-21. (canceled)

22. The method of claim 1, wherein selecting CTCs by assessing nuclear area comprises determining pixel size for each CTC and applying a pre-determined threshold for exclusion or comprises determining nuclear diameter and/or determining DAPI concentration and its standard deviation.

23. (canceled)

24. The method of claim 1, wherein step (d) comprises assessing all abnormalities or gains only.

25. The method of claim 1, further comprising obtaining said sample.

26. A method of determining the level of circulating tumor cells (CTCs) in a sample containing blood cells from a patient by:
(a) selecting CTCs from a first blood sample by assessing nuclear size and/or circularity and/or DAPI concentration;
(b) contacting the selected cells with labeled nucleic acid probes for 3p22.1, 10q22.3, chromosome 10 centromeric (cep10) and chromosome 3 centromeric (cep3);
(c) detecting fluorescence in situ hybridization from cells; and
(d) quantifying CTCs based on hybridization to all four labeled nucleic acid probes.

27. The method of claim 26, wherein the presence of CTCs equaling 4 or more in the sample is indicative of cancer, such as wherein the sample is a 5 ml sample of a separated buffy coat layer.

28. The method of claim 26, wherein the presence of CTCs in the blood sample, in the presence of an indeterminate nodule of greater than 3 mm in the lung, is indicative of cancer.

29. The method of claim 26, wherein the presence of CTCs in the blood sample is indicative of lung cancer.

30-32. (canceled)

33. The method of claim 26, wherein a higher level of CTCs in the sample, as compared to a control or predetermined number of CTCs from a non-aggressive form of cancer, is indicative of an aggressive form of cancer and/or a poor cancer prognosis.

34-37. (canceled)

38. The method of claim 26, further comprising:
determining the level of CTCs in a second sample from the patient after treatment is effected and
comparing the level of CTCs in the first sample with the level of CTCs in the second sample to assess a change,
thereby monitoring treatment.

39-42. (canceled)

43. The method of claim 26, wherein a higher level of CTCs in the sample as compared to a predetermined control for a given stage is indicative of a more advanced stage of cancer, and a lower level of CTCs in the sample as compared to a control for a given stage is indicative of a less advanced stage of cancer.

44-51. (canceled)

52. The method of claim 43, further comprising obtaining a patient sample or reporting the level of CTCs.

53. (canceled)

54. The method of claim 43, further comprising treating the cancer if the level of CTCs is indicative of a more advanced stage of cancer.

55-57. (canceled)
